(19) United States
(12) Patent Application Publication
(10) Pub. No.: US 2026/0167996 A1
(43) Pub. Date: Jun. 18, 2026

(54) ALGAE WITH MODIFIED LIPASE GENE AND DIACYLGLYCEROL ACYLTRANSFERASE GENE, AND METHOD OF PRODUCING TRIACYLGLYCEROL USING THE SAME

(71) Applicants: PHYTOLIPID TECHNOLOGIES CO., LTD., Yokohama-shi (JP); HIROSHIMA UNIVERSITY, Higashihiroshima-shi (JP); MAZDA MOTOR CORPORATION, Aki-gun (JP)

(72) Inventors: Hiroyuki OHTA, Tokyo (JP); Mie SHIMOJIMA, Tokyo (JP); Masako IWAI, Yokohama-shi (JP); Natsuko KATO, Yokohama-shi (JP); Atsushi SAKAMOTO, Higashihiroshima-shi (JP); Takashi YAMAMOTO, Higashihiroshima-shi (JP); Kumiko OKAZAKI, Higashihiroshima-shi (JP); Tomokazu KURITA, Higashihiroshima-shi (JP); Shinichiro MAEDA, Aki-gun (JP)

(73) Assignees: PHYTOLIPID TECHNOLOGIES CO., LTD., Yokohama-shi (JP); HIROSHIMA UNIVERSITY, Higashihiroshima-shi (JP); MAZDA MOTOR CORPORATION, Aki-gun (JP)

(21) Appl. No.: 19/414,592

(22) Filed: Dec. 10, 2025

(30) Foreign Application Priority Data
Dec. 13, 2024 (JP) 2024-218986

(51) Int. Cl. C12P 7/6463; C12N 1/12; C12N 9/10; C12N 9/20; C12N 15/79; C12R 1/89

(52) U.S. Cl. CPC C12P 7/6463; C12N 1/12; C12N 9/1029; C12N 9/20; C12N 15/79; C12R 2001/89; C12Y 203/0102; C12Y 301/01003

(57) ABSTRACT

Provided is means for achieving increase in the amounts of triacylglycerol accumulated in algae with high lipid accumulation. An alga having the following characteristics (1) and (2): (1) having reduced expression of a class 3 lipase gene; and (2) having an exogenous diacylglycerol acyltransferase gene introduced or having enhanced expression of an endogenous diacylglycerol acyltransferase gene.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 5, 2025, is named “FP-349 sequence.xml” and is 83,331 bytes in size. The sequence listing contained in this .XML file is part of the specification and is hereby incorporated by reference herein in its entirety.

### TECHNICAL FIELD

The present invention relates to an alga that accumulates a large amount of triacylglycerol, and a method of producing triacylglycerol using the same.

### BACKGROUND ART

Reduction in nutrient salts causes growth reduction and accumulation of lipid such as triacylglycerol (TAG) in most plants and algae. The biomass of algae is larger than that of plants, and algae are advantageous in biofuel production and useful-lipid production (Non Patent Literature 1). Research is globally ongoing to produce industrial and food products including biofuels from alga-derived TAG as a raw material. However, the current production cost is disadvantageously high for the progress of industrialization. Accordingly, further technical development is essential for production cost reduction.

The amount of TAG accumulated is regulated through lipid degradation and lipid synthesis. Lipase is known as a protein involved in TAG degradation, and diacylglycerol acyltransferase (DGAT) is known as a protein involved in TAG synthesis. The eustigmatophyte Nannochloropsis (hereinafter, referred to as “Nannochloropsis”) is an ultra-microalga, and reported to accumulate a certain amount of TAG in normal culture, and accumulate a larger amount of TAG under nutrient-deficient conditions, wherein the fatty acid composition of TAG is simple and suitable for fuels (Non Patent Literature 1, Non Patent Literature 2). Nannochloropsis allows seawater culture and high-density culture, thus enabling low-cost production, and is characterized in that gene modification techniques have been already established therefor (Non Patent Literature 3, Non Patent Literature 4). Both TGL and SDP1, major TAG lipases known in budding yeasts and higher plants, have a Patatin like domain (Non Patent Literature 5, Non Patent Literature 6, Non Patent Literature 7). It has been reported that if the expression level of an AtSDP1 homolog gene in a diatom (Phaeodactylum tricornutum) is reduced to 20% to 40% thereof by means of RNAi, the amount of TAG doubles (Non Patent Literature 8). Nannochloropsis has lipases of the same domain structure, TGL1 and TGL2. A TGL1/TGL2 double mutant has been produced, and found to exhibit increase in the amount of TAG accumulated on day 2 in the early stage of culture (Non Patent Literature 9) However, the increase in the amount of TAG accumulated was not found in the late stage of culture or under nutrient-deficient conditions, and hence the TAG degradation in Nannochloropsis is expected to be mainly caused by an unknown lipase other than TGL1 and TGL2. Previously, the present inventors have reported that if a DGAT2 gene derived from Chlamydomonas reinhardtii, which is involved in TAG synthesis, is introduced into Nannochloropsis and overexpressed under nutrient-deficient conditions, the amount of TAG accumulated increases (Patent Literature 1). However, there is no example of producing an algal strain having functional suppression of a protein involved in TAG degradation and at the same time having enhanced expression of a protein involved in TAG synthesis by using the alga with high lipid production, Nannochloropsis.

### CITATION LIST

### Patent Literature

[Patent Literature 1] International Publication No. WO 2015/137449

### Non Patent Literature

[Non Patent Literature 1] Plant J. 54, 621-639, (2008)
[Non Patent Literature 2] Biotechnol. Bioeng. 102, 100-112, (2009)
[Non Patent Literature 3] Proc. Natl. Acad. Sci. USA 108, 21265-21269, (2011)
[Non Patent Literature 4] Genes Cells 25, 695-702, (2020)
[Non Patent Literature 5] Plant Cell 18, 665-675, (2006)
[Non Patent Literature 6] J. Biol. Chem. 278, 23317-23323, (2003)
[Non Patent Literature 7] J. Biol. Chem. 280, 37301-37309, (2005)
[Non Patent Literature 8] Biochim. Biophys. Acta 1861, 239-248, (2016)
[Non Patent Literature 9] Biochim. Biophys. Acta 1864, 1185-1193, (2019)

### SUMMARY OF INVENTION

### Technical Problem

Nannochloropsis is an alga with higher lipid accumulation than other algae, and further technical development is desired for production cost reduction. The present invention has been made against such background, and an object of the present invention is to provide means for causing increase in the amounts of TAG accumulated in algae with high lipid accumulation such as Nannochloropsis.

### Solution to Problem

Gene-disrupted strains of Nannochloropsis in which TGL1 and TGL2, which are homologs of the main TAG lipase SDP1 in plants, have been disrupted do not provide increased TAG production at practical levels (Biochim. Biophys. Acta 1864, 1185-1193, (2019)). With this in mind, the present inventors have diligently conducted a study to achieve the above object, and newly focused on class 3 lipase (Pfam #PF01764) as a TAG lipase. Its structure represents a domain having an α/β hydrolase fold such as feruloyl esterase A of Aspergillus niger (J. Mol. Biol. 338, 495-506, (2004)), the triacylglycerol lipase OBL1 of Arabidopsis thaliana (New Phytol 217, 1062-1076, (2018).), and human diacylglycerol lipase α (Proc Natl Acad Sci USA 113, 26-33, (2016).). Pfam domain search found 23 class 3 lipases from Nannochloropsis. For No3LIP7 and No3LIP14 among them, gene-disrupted strains were produced by genome editing; for No3LIP6 and No3LIP10, gene-disrupted strains were produced by homologous recombination. The No3LIP7, No3LIP14, No3LIP6, and No3LIP10 gene-disrupted strains successfully exhibited increase in the amount of TAG accumulated in the late stage of culture. Enhancement of expression of genes involved in TAG synthesis was performed for further increased TAG accumulation. Focusing on DGAT1 and DGAT2 of fish feeding on algae as genes to introduce, the present inventors obtained two genes, CodDGAT1 and CodDGAT2, by artificial synthesis. These genes were each sandwiched between an LDSP promoter region and LDSP terminator region of Nannochloropsis to produce constructs for forced expression, and the constructs were each introduced into the No3LIP14 gene-disrupted strain. As a result, further increase in the amount of TAG accumulated was successfully achieved. The present invention solves the problem shown above by combining disruption of novel lipase genes and high DGAT gene expression.

The present invention provides the following [1] to [8].
[1] An alga having the following characteristics (1) and (2):
   (1) having reduced expression of a class 3 lipase gene; and
   (2) having an exogenous diacylglycerol acyltransferase gene introduced or having enhanced expression of an endogenous diacylglycerol acyltransferase gene.
[2] The alga according to [1], wherein the alga is an alga belonging to the genus Nannochloropsis.
[3] The alga according to [1], wherein the class 3 lipase gene is a gene encoding the following protein (a), (b), or (c):
   (a) a protein consisting of an amino acid sequence set forth in SEQ ID NO: 2, 4, 6, or 8;
   (b) a protein consisting of an amino acid sequence derived by substitution, addition, or deletion of 1 to 50 amino acid residues from an amino acid sequence set forth in SEQ ID NO: 2, 4, 6, or 8 and having lipase activity; and
   (c) a protein consisting of an amino acid sequence having a homology of 40% or more with an amino acid sequence set forth in SEQ ID NO: 2, 4, 6, or 8 and having lipase activity.
[4] The alga according to [1], wherein the exogenous diacylglycerol acyltransferase gene is a gene encoding the following protein (a), (b), or (c):
   (a) a protein consisting of an amino acid sequence set forth in SEQ ID NO: 10 or 12;
   (b) a protein consisting of an amino acid sequence derived by substitution, addition, or deletion of 1 to 50 amino acid residues from an amino acid sequence set forth in SEQ ID NO: 10 or 12 and having diacylglycerol acyltransferase activity; and
   (c) a protein consisting of an amino acid sequence having a homology of 40% or more with an amino acid sequence set forth in SEQ ID NO: 10 or 12 and having diacylglycerol acyltransferase activity.
[5] The alga according to [1], wherein the endogenous diacylglycerol acyltransferase gene is a gene encoding the following protein (a), (b), or (c):
   (a) a protein consisting of an amino acid sequence set forth in SEQ ID NO: 14, 16, 18, 20, 22, or 24;
   (b) a protein consisting of an amino acid sequence derived by substitution, addition, or deletion of 1 to 50 amino acid residues from an amino acid sequence set forth in SEQ ID NO: 14, 16, 18, 20, 22, or 24 and having diacylglycerol acyltransferase activity; and
   (c) a protein consisting of an amino acid sequence having a homology of 60% or more with an amino acid sequence set forth in SEQ ID NO: 14, 16, 18, 20, 22, or 24 and having diacylglycerol acyltransferase activity.
[6] The alga according to [1], wherein, when cultured under phosphorus-deficient conditions, the alga produces triacylglycerol having a lower rate of oleic acid and a higher rate of palmitic acid than a wild strain produces.
[7] A method of producing triacylglycerol, comprising: culturing the alga according to any one of [1] to [6] to allow the alga to produce triacylglycerol, and collecting the triacylglycerol produced is.
[8] The method of producing triacylglycerol according to [7], wherein the culturing the alga occurs under phosphorus-deficient conditions.

### Advantageous Effects of Invention

The present invention provides a novel alga. This alga accumulates a large amount of TAG, and therefore is useful for production of TAG. The TAG produced by the alga has a lower rate of oleic acid (C18:1) and higher rate of palmitic acid (C16:0) than TAG produced by the wild strain has, and therefore is suitable as a raw material of biofuels.

### BRIEF DESCRIPTION OF DRAWINGS

FIG. 1 is a diagram showing constructs for No3LIP6 and No3LIP10 gene disruption.

FIG. 2 is a series of graphs showing the growth of No3LIP7 gene-disrupted strains. * indicates the presence of significant difference between WT and No3LIP7, >* indicates the presence of significant difference between the upper side and the lower side, and ** indicates the presence of significant difference between any two of the three (p<0.05, Tukey test, n=4, error bar: se).

FIG. 3 is a series of graphs showing the amounts of TAG accumulated in No3LIP7 gene-disrupted strains (p<0.05, Tukey test, n=4, error bar: se).

FIG. 4 is a series of graphs showing the fatty acid compositions in TAG in No3LIP7 gene-disrupted strains (p<0.05, Tukey test, n=4, error bar: se).

FIG. 5 is a series of graphs showing the growth of No3LIP14 gene-disrupted strains. * indicates the presence of significant difference between WT and No3LIP14, and >* indicates the presence of significant difference between the upper side and the lower side (p<0.05, Tukey test, n=4, error bar: se).

FIG. 6 is a series of graphs showing the amounts of TAG accumulated in No3LIP14 gene-disrupted strains (p<0.05, Tukey test, n=4, error bar: se).

FIG. 7 is a series of graphs showing the fatty acid compositions in TAG in No3LIP14 gene-disrupted strains (p<0.05, Tukey test, n=4, error bar: se).

FIG. 8 is a series of graphs showing the growth of No3LIP6 gene-disrupted strains and the amounts of TAG accumulated therein (p<0.05, Tukey test, n=4, error bar: se).

FIG. 9 is a series of graphs showing the growth of No3LIP10 gene-disrupted strains and the amounts of TAG accumulated therein (p<0.05, Tukey test, n=4, error bar: se).

FIG. 10 is a diagram showing constructs for enhancing the TAG production system.

FIG. 11 is a series of graphs showing the growth of codDGAT1/LIP14 and codDGAT2/LIP14. ** indicates the presence of significant difference between WT and any other, * indicates the presence of significant difference between codDGAT1/LIP14B1-4 and any other, and >* indicates the presence of significant difference between the upper side and the lower side (p<0.05, Tukey test, error bar: se, n=3 for normal medium, n=4 for phosphorous-deficient medium).

FIG. 12 is a series of graphs showing the amounts of TAG accumulated in codDGAT1/LIP14 and codDGAT2/LIP14 (p<0.05, Tukey test, error bar: se, n=3 for normal medium, n=4 for phosphorous-deficient medium).

FIG. 13 is a series of graphs showing the fatty acid compositions in TAG in codDGAT1/LIP14 and codDGAT2/LIP14 (p<0.05, Tukey test, error bar: se, n=4).

FIG. 14 is a series of diagrams showing light/temperature conditions for high-density aerobic culture. A. A light/temperature control program used for culture. Solid lines represent light intensity, and dashed lines represent temperature. B. Culture conditions for preculture and main culture.

FIG. 15 is a series of graphs for comparison of the amounts of biomass of the wild strain, the parent strain, codDGAT1/LIP14, and codDGAT2/LIP14. A. Temporal variations of amounts of biomass per culture solution. B. Temporal variations of amounts of biomass per cell. * indicates the presence of significant difference between WT and CodDGAT2, ** indicates the presence of significant difference between WT and CodDGAT1 and between WT and CodDGAT2. C. Amounts of biomass per culture solution on day 7 after the initiation of culture. D. Amounts of biomass per cell on day 7 after the initiation of culture. n=3, error bar: se. p<0.05 (Tukey test).

FIG. 16 is a series of graphs for comparison of the amounts of TAG accumulated in the wild strain, the parent strain, codDGAT1/LIP14, and codDGAT2/LIP14. A. Temporal variations of amounts of TAG accumulated per culture solution. * indicates the presence of significant difference between WT and CodDGAT2. B. Amounts of TAG accumulated per culture solution on day 10 after the initiation of culture. n=3, error bar: se. p<0.05 (Tukey test).

FIG. 17 is a series of graphs showing the fatty acid compositions in TAG in the wild strain, the parent strain, codDGAT1/LIP14, and codDGAT2/LIP14 (p<0.05, Tukey test, error bar: se, n=3).

### DESCRIPTION OF EMBODIMENTS

The following describes the present invention in detail.

The alga of the present invention is preferably an alga belonging to the genus Nannochloropsis, but may be any other alga. Examples of the algae belonging to the genus Nannochloropsis can include Nannochloropsis oceanica, Nannochloropsis gaditana, Nannochloropsis salina, Nannochloropsis oculata, Nannochloropsis atomus, Nannochloropsis maculata, Nannochloropsis granulata, Nannochloropsis limnetica, Nannochloropsis maritima, and Nannochloropsis australis.

The alga of the present invention has the following two characteristics.

The first characteristic is having reduced expression of a class 3 lipase gene. Class 3 lipase has a domain having an α/β hydrolase fold. Accordingly, the class 3 lipase gene in the alga can be identified on the basis of that domain (e.g., accession number in Pfam database: PF01764). The class 3 lipase gene is preferably for class 3 triacylglycerol lipase.

Specific examples of the class 3 lipase gene can include No3LIP7, No3LIP14, No3LIP6, No3LIP10, and genes corresponding to those genes in individual algal species. The nucleotide sequences of No3LIP7, No3LIP14, No3LIP6, and No3LIP10 are as set forth in SEQ ID NOs: 1, 3, 5, and 7, respectively, and the amino acid sequences of proteins encoded by those genes are as set forth in SEQ ID NOs: 2, 4, 6, and 8, respectively. Examples of genes corresponding to No3LIP7, No3LIP14, No3LIP6, or No3LIP10 can include a gene encoding (b) a protein consisting of an amino acid sequence derived by substitution, addition, or deletion of 1 to 50 amino acid residues from an amino acid sequence set forth in SEQ ID NO: 2, 4, 6, or 8 and having lipase activity, or (c) a protein consisting of an amino acid sequence having a homology of 40% or more with an amino acid sequence set forth in SEQ ID NO: 2, 4, 6, or 8 and having lipase activity.

The number of amino acid residues to be substituted, added, or deleted in the protein (b) may be any number of 1 to 50 without limitation, and can be preferably about 1 to 30, more preferably about 1 to 10, even more preferably about 1 to 5, and particularly preferably about 1, 2, 3, or 4.

The homology of the protein (c) with an amino acid sequence set forth in SEQ ID NO: 2, 4, 6, or 8 may be any value of 40% or more without limitation, and is preferably 50% or more, more preferably 60% or more, even more preferably 70% or more, and particularly preferably 80% or more. The value of homology may be further higher, and can be, for example, 90% or more, 95% or more, 97% or more, 98% or more, or 99% or more. The homology of an amino acid sequence can be calculated by using the program BLASTP provided by NCBI (National Center of Biotechnology Information).

Blast search conducted for the amino acid sequences set forth in SEQ ID NOs: 2, 4, 6, and 8 found the presence of a protein having a homology of approximately 70% with that of Nannochloropsis gaditana for SEQ ID NO: 2 (No3LIP7), the presence of a protein having a homology of approximately 40% with those of Nannochloropsis gaditana and Nannochloropsis salina for SEQ ID NO: 4 (No3LIP14), the presence of a protein having a homology of approximately 80% with those of Nannochloropsis gaditana and Nannochloropsis salina for SEQ ID NO: 6 (No3LIP6), and the presence of a protein having a homology of approximately 60% with that of Nannochloropsis gaditana and a homology of approximately 70% with that of Nannochloropsis salina for SEQ ID NO: 8 (No3LIP10). From this, genes encoding the protein (c) (genes that have a homology of 40% or more at amino acid levels) are expected to include genes corresponding to No3LIP7, No3LIP14, No3LIP6, and No3LIP10 in algae belonging to the genus Nannochloropsis.

While the alga of the present invention has reduced expression of a class 3 lipase gene, the expression “reduced expression of a class 3 lipase gene” means that the expression level of a class 3 lipase gene is lower than that in the wild strain, and a case without any expression of a class 3 lipase gene is also included in the meaning. The reduced expression may be caused by a procedure that affects the gene in the genome (e.g., gene modification by genome editing, mutation by radiation), or by a procedure that does not affect the gene in the genome (e.g., suppression of gene expression by an RNAi method or an antisense method).

Examples of modification of a class 3 lipase gene can include gene knockout (gene disruption), introduction of a mutation into a protein-coding region, and introduction of a mutation into an expression-controlling region.

The second characteristic is having an exogenous DGAT gene introduced or having enhanced expression of an endogenous DGAT gene. While the second characteristic may be any of introduction of an exogenous gene and enhanced expression of an endogenous gene, algae with enhanced expression of an endogenous DGAT gene do not correspond to recombinant organisms in normal cases, allowing outdoor culture, and thus is preferable in this regard.

For DGAT, there are two isozymes: DGAT1 and DGAT2. Any of them may be used in the present invention. Plant-derived and animal-derived DGAT genes are known, and any of those DGAT genes may be used as the exogenous DGAT gene.

Specific examples of the exogenous DGAT gene can include CodDGAT1, CodDGAT2, and genes similar to those genes. The nucleotide sequences of CodDGAT1 and CodDGAT2 are as set forth in SEQ ID NOs: 9 and 11, respectively, and the amino acid sequences of proteins encoded by those genes are as set forth in SEQ ID NOs: 10 and 12, respectively. Examples of genes similar to CodDGAT1 or CodDGAT2 can include a gene encoding (b) a protein consisting of an amino acid sequence derived by substitution, addition, or deletion of 1 to 50 amino acid residues from an amino acid sequence set forth in SEQ ID NO: 10 or 12 and having DGAT activity, or (c) a protein consisting of an amino acid sequence having a homology of 40% or more with an amino acid sequence set forth in SEQ ID NO: 10 or 12 and having DGAT activity.

The number of amino acid residues to be substituted, added, or deleted in the protein (b) may be any number of 1 to 50 without limitation, and can be preferably about 1 to 30, more preferably about 1 to 10, even more preferably about 1 to 5, and particularly preferably about 1, 2, 3, or 4.

The homology of the protein (c) with an amino acid sequence set forth in SEQ ID NO: 10 or 12 may be any value of 40% or more without limitation, and is preferably 50% or more, more preferably 60% or more, even more preferably 70% or more, and particularly preferably 80% or more. The value of homology may be further higher, and can be, for example, 90% or more, 95% or more, 97% or more, 98% or more, or 99% or more.

Since most algae are known to have an endogenous DGAT gene, the expression of such a DGAT gene can be suitably enhanced in the present invention. Such genes as DGAT1A, DGAT2A, and DGAT2B are known as endogenous DGAT genes in algae belonging to the genus Nannochloropsis. The nucleotide sequences of DGAT1A, DGAT2A, and DGAT2B of Nannochloropsis oceanica NIES-2145 strain are as set forth in SEQ ID NOs: 13, 15, and 17, respectively, and the amino acid sequences of proteins encoded by those genes are as set forth in SEQ ID NOs: 14, 16, and 18, respectively. The nucleotide sequences of DGAT1A, DGAT2A, and DGAT2B of Nannochloropsis oceanica IMET1 strain are published (Wei, H. et al., Biotechnol Biofuels 10, 174 (2017), accession numbers in GenBank: KY073295.1, KX867956.1, and KX867957.1, respectively), and as set forth in SEQ ID NOs: 19, 21, and 23, respectively, and the amino acid sequences of proteins encoded by those genes are as set forth in SEQ ID NOs: 20, 22, and 24, respectively. Genes of the NIES-2145 strain or IMET1 strain can be used in the present invention, whereas genes similar to those genes may be used. Examples of such similar genes can include a gene encoding (b) a protein consisting of an amino acid sequence derived by substitution, addition, or deletion of 1 to 50 amino acid residues from an amino acid sequence set forth in SEQ ID NO: 14, 16, 18, 20, 22, or 24 and having DGAT activity, or (c) a protein consisting of an amino acid sequence having a homology of 60% or more with an amino acid sequence set forth in SEQ ID NO: 14, 16, 18, 20, 22, or 24 and having DGAT activity.

The number of amino acid residues to be substituted, added, or deleted in the protein (b) may be any number of 1 to 50 without limitation, and can be preferably about 1 to 30, more preferably about 1 to 10, even more preferably about 1 to 5, and particularly preferably about 1, 2, 3, or 4.

The homology of the protein (c) with an amino acid sequence set forth in SEQ ID NO: 14, 16, 18, 20, 22, or 24 may be any value of 60% or more without limitation, and is preferably 70% or more, more preferably 80% or more, even more preferably 90% or more, and particularly preferably 95% or more. The value of homology may be further higher, and can be, for example, 97% or more, 98% or more, or 99% or more.

Any method may be used for enhancing the expression of the endogenous DGAT gene without limitation, and examples can include a method of replacing a promoter or terminator of the endogenous DGAT gene with a promoter or terminator that enables high-level protein expression. The promoter and terminator that enable high-level protein expression are not limited, whereas use of an endogenous promoter and terminator is preferable because if an exogenous promoter or terminator is used, the resulting alga corresponds to a recombinant organism. The promoter and terminator that enable high-level protein expression may be a promoter and terminator that enable high-level protein expression under normal conditions, or a promoter and terminator that enable high-level protein expression only under special conditions such as phosphorus-deficient conditions.

Specific examples of the promoter that enables high-level protein expression can include an LDSP promoter derived from algae belonging to the genus Nannochloropsis, and specific examples of the terminator that enables high-level protein expression can include an LDSP terminator derived from algae belonging to the genus Nannochloropsis. The nucleotide sequences of the LDSP promoter and terminator derived from algae belonging to the genus Nannochloropsis are as set forth in SEQ ID NOs: 25 and 26, respectively. The promoter and terminator to be used are not limited to those, and, for example, a promoter and terminator of an LHC gene, a promoter and terminator of an FCP gene, a promoter and terminator of a VCP1 gene, a promoter and terminator of an elongation factor 1-alpha gene, a promoter and terminator of an Actin gene, a promoter and terminator of a Tubulin beta gene, or a promoter and terminator of a Tubulin alpha gene may be used as a promoter and terminator that enable high-level protein expression under normal conditions. Alternatively, a promoter and terminator of an SQD2 gene may be used as a promoter and terminator that enable high-level protein expression under phosphorus-deficient conditions.

Examples of the characteristics of the alga of the present invention can include: 1) accumulating a large amount of TAG in cells under any of normal culture conditions and phosphorus-deficient conditions; and 2) producing TAG having a lower rate of oleic acid (C18:1) and a higher rate of palmitic acid (C16:0) when cultured under phosphorus-deficient conditions than the wild strain produces.

The method of producing TAG of the present invention comprises: culturing the alga of the present invention to allow the alga to produce TAG, and collecting the TAG produced.

The alga of the present invention may be cultured under normal conditions (under conditions without phosphorus deficiency), but is preferably cultured under phosphorus-deficient conditions to increase the amount of TAG accumulated. For matters other than phosphorus, appropriate culture conditions fitting with the type of the alga can be selected. For example, in the case that the alga to be cultured is an alga belonging to the genus Nannochloropsis, the medium to be used can be F2N medium, HD medium, or a medium obtained by removing phosphorus from any of the two, the culture temperature can be about 15 to 30° C., and the light intensity during culture can be 10 to 2000 μmol photons/m²/sec.

Examples of methods for collecting TAG produced in the alga can include methods that are conventionally used in recovering TAG accumulated in cells such as a method of recovering TAG by drying, freezing, crushing, filtering, centrifugation, or solvent extraction of algal cells.

### EXAMPLES

The following describes the present invention in more detail with examples, but the present invention is not limited to those examples.

### Experimental Materials

The eustigmatophyte Nannochloropsis NIES-2145 (hereinafter, referred to as “N.2145”) was used. This algal strain is available from National Institute for Environmental Studies, Japan (http://www.nies.go.jp/).

### Genes Used

The transcript data of Nannochloropsis oceanica CCMP1779 v2.0 (https://phycocosm.jgi.doe.gov/Nanoce1779_2/Nanoce1779_2. home.html) was searched for novel lipase genes having the Lipase class 3 domain (Pfam #PF01764), and 23 genes were selected as candidates. From the expression profiles of them, No3LIP7, No3LIP14, No3LIP6, and No3LIP10 were picked up.

The sequences of codDGAT1 and codDGAT2 were determined from those from Atlantic cod (Gadus morhua), which are published and available by querying Human DGAT1 and Human DGAT2 (https://www.ncbi.nlm.nih.gov/datasets/genome/GCF_902167405.1/), and modified products thereof to have codons of Nannochloropsis were obtained through artificial synthesis by Eurofins Scientific SE.

The sequences of the genes are listed in a sequence listing. The sequences of No3LIP7, No3LIP14, No3LIP6, No3LIP10, codDGAT1, and codDGAT2 are set forth in SEQ ID NOs: 1, 3, 5, 7, 9, and 11, respectively.

### Experimental Operations

### 1. Culture Conditions

Except for high-density aerobic culture, F2N medium was used as a medium for normal solution culture in culture of N.2145. For plate culture, F2N medium with addition of 0.8% of Agar was used.

In 100 mL of ion-exchanged water, 440 mg of Na₂EDTA·2H₂O, 316 mg of FeCl₃·6H₂O, 1.2 mg of CoSO₄·7H₂O, 2.1 mg of ZnSO₄·7H₂O, 18 mg of MnCl₂·4H₂O, 0.7 mg of CuSO₄·5H₂O, and 0.7 mg of Na₂MoO₄·2H₂O were dissolved, and the resultant was stored in advance as f/2 metal at 4° C. In 900 mL of ion-exchanged water, 121.14 g of tris(hydroxymethyl)aminomethane was dissolved, the pH was adjusted to 7.6 with HCl, the volume was adjusted to 1 L, and the resultant was stored in advance as 1 M Tris-HCl (pH 7.6) at 4° C. In 98.5 mL of artificial seawater, 7.5 mg of NaNO₃, 26.745 mg of NH₄Cl, 3 mg of NaH₂PO₄·2H₂O, 0.25 μg of Vitamin B₁₂, 0.25 μg of Biotin, 50 μg of Thiamine HCl, 0.5 mL of f/2 metal, and 1 mL of 1 M Tris-HCl (pH 7.6) were dissolved, and the resultant was filter-sterilized and used as F2N medium. The artificial seawater used was Daigo's Artificial Seawater SP (FUJIFILM Wako Pure Chemical Corporation). F2N medium removed of NaH₂PO₄ was used as phosphorus-deficient medium. Only when a strain obtained by introducing codDGAT1 or codDGAT2 into No3LIP14 #6B-6 was cultured with normal medium, the strain was cultured through gyratory culture at 30 μmol photons/m²/sec, 23° C., and 120 min⁻¹, and culture was performed for three samples for each case. The other strains were cultured through gyratory culture with each liquid medium at 50 to 60 μmol photons/m²/sec, 23° C., and 120 min⁻¹. Culture was performed for four samples for each case. The cell concentration of each strain at the initiation of culture was set to 3×10⁶ cells/mL. A CellDrop BF (DeNovix) was used for counting the numbers of cells.

For high-density aerobic culture, HD medium was used as a medium for normal solution culture.

In 1 L of ion-exchanged water, 222 mg of ZnSO₄·7H₂O, 79 mg of CuSO₄·5H₂O, 15 mg of MoO₃, 2.86 g of H₃BO₃, and 1.81 g of MnCl₂·4H₂O were dissolved, and the resultant was stored in advance as A-5 stock at 4° C. A product obtained by dissolving 2.5 g of KNO₃, 0.25 g of Na₂HPO₄, 0.075 g of Fe-EDTA, and 5 mL of A-5 stock in 500 mL of ion-exchanged water and a product obtained by dissolving Daigo's Artificial Seawater SP (FUJIFILM Wako Pure Chemical Corporation) in 500 mL of ion-exchanged water were each sterilized in an autoclave and then mixed together, to which 2 mL of vitamin mix solution was added, and the resultant was used as HD medium. The vitamin mix solution had been prepared by dissolving 0.6 μg of Vitamin B₁₂, 0.3 μg of Biotin, and 60 μg of Thiamine HCl in 10 mL of ion-exchanged water 10 mL, and filter-sterilized before use. HD medium removed of Na₂HPO₄ was used as phosphorus-deficient medium, and HD medium with the amount of Na₂HPO₄ reduced to 0.125 g was used as phosphorus ½ medium. In normal culture, 5 mL of culture solution cultured for 7 days was added to 50 mL of HD medium, and aeration was performed at 700 μmol photons/m²/sec and 25° C. with 2% CO₂ at 15 mL/min.

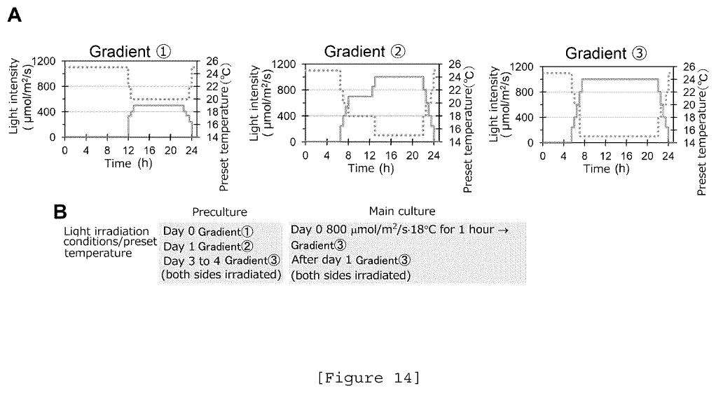

Cells subjected to normal culture for 7 days were successively seeded in 500 mL of phosphorus 1/2 medium to 1×10⁸ cells/mL, and precultured under the light irradiation conditions at the preset temperature of the incubator as shown in FIG. 14 for 4 days, while aeration was performed with 2% CO₂ at 450 mL/min. Thereafter, cells were successively seeded in 500 mL of phosphorus-deficient medium to 2×10⁸ cells/mL, and cultured under the light irradiation conditions at the preset temperature of the incubator as shown in FIG. 14 for 10 days, while aeration was performed with 2% CO₂ at 450 mL/min. Culture under phosphorus-deficient conditions was performed for three samples for each case. The flow cytometer RF-500 (SYSMEX CORPORATION) was used for counting the numbers of cells.

### 2. Lipid Extraction

Except for samples subjected to high-density aerobic culture, 1 mL of culture solution cultured with normal medium or phosphorus-deficient medium was collected on each day, and cryopreserved at −80° C.

To the frozen cells, 1.5 mL of chloroform and 3 mL of methanol were added, and the cells were left to stand at room temperature for 1 hour while suspended every 10 minutes. The resultant was centrifuged with a swing rotor at 1000×g for 5 minutes, and 5.5 mL of the supernatant was recovered as a first extract. To the precipitate, 0.26 mL of 1% (w/v) KCl, 0.4 mL of chloroform, and 0.8 mL of methanol were added to form a suspension, which was then centrifuged with a swing rotor at 1000×g for 5 minutes, and 1.46 mL of the supernatant was recovered as a second extract. The first extract and the second extract were combined, 2.16 mL of 1% (w/v) KCl and 1.9 mL of chloroform were further added to form a suspension, which was then centrifuged with a swing rotor at 1000×g for 5 minutes, and the lower layer was recovered as a lipid extract. The lipid extract was dried, dissolved in chloroform:methanol=2:1, and stored at −20° C.

For samples subjected to high-density aerobic culture, 10 mL of culture solution cultured with phosphorus-deficient medium was collected on each day, and cells were recovered and cryopreserved at −80° C.

The frozen cells were suspended in 0.8 mL of ion-exchanged water, 1 mL of chloroform and 2 mL of methanol were added, and the resultant was stirred and left to stand at room temperature for 1 hour. Thereto, 1 mL of chloroform and 1 mL of ion-exchanged water were added to form a suspension, which was centrifuged with a swing rotor at 1000×g for 5 minutes, and the water/methanol layer (upper layer) was removed and the chloroform layer (lower layer) was transferred to an additional glass test tube. Meanwhile, 1.5 mL of chloroform was added to the original glass test tube to form a suspension. This original test tube with the suspension and the additional test tube to which the chloroform layer had been transferred were together centrifuged with a swing rotor at 1000×g for 5 minutes. After the centrifugation, the chloroform layer in the additional test tube was transferred to another test tube the weight of which had been measured. The chloroform layer in the original test tube was recovered, centrifuged with a swing rotor at 1000×g for 5 minutes and recovered, and combined with the previous chloroform extract to give a lipid extract. This lipid extract was dried with a vacuum concentrator, dissolved in chloroform to 10 mg/mL, and then stored at −20° C.

### 3. Lipid Analysis

Except for samples subjected to high-density aerobic culture, lipid extract was spotted on a thin-layer silica plate, and developed with developing solution of 160 mL of hexane, 40 mL of diethyl ether, and 4 mL of acetic acid for 45 minutes. TAG was confirmed with 0.001% (w/v) primuline under UV irradiation. Silica in the part having TAG thereon was scraped, and 50 μL of 1 mM heneicosanoic acid and 500 μL of 1.5 M hydrochloric acid/methanol were added to form a suspension, which was then left to stand at 85° C. for 1 hour for methyl-esterification of fatty acids. Thereto, 500 μL of hexane was added to form a suspension, which was then centrifuged with a swing rotor at 1000×g for 5 minutes, and methyl-esterified fatty acids in the upper layer were recovered. Again, 500 μL of hexane was added to the lower layer to form a suspension, which was then centrifuged with a swing rotor at 1000×g for 5 minutes, and the upper layer was recovered. The methyl-esterified fatty acids recovered were dried, and then dissolved in 100 μL of hexane, giving a gas chromatography sample. Gas chromatography was performed by using a SHIMADZU GC-2030 equipped with an HR-SS-10 (0.25φ×25 m) (SHINWA CHEMICAL INDUSTRIES, LTD.).

For samples subjected to high-density aerobic culture, lipid extract was spotted on a thin-layer silica plate, and developed with developing solution of 160 mL of hexane, 40 mL of diethyl ether, and 4 mL of acetic acid for 35 minutes. TAG was confirmed with 0.001% (w/v) primuline under UV irradiation. Silica in the part having TAG thereon was scraped, and 10 μL of 5 mM heneicosanoic acid and 2.5 mL of 1.5 M hydrochloric acid/methanol were added to form a suspension, which was then left to stand at 85° C. for 2.5 hours for methyl-esterification of fatty acids. Thereto, 2.5 mL of hexane was added to form a suspension, which was then left to stand, and methyl-esterified fatty acids in the upper layer were recovered. The methyl-esterified fatty acids recovered were dried, and then dissolved in 100 μL of hexane, giving a gas chromatography sample. Gas chromatography was performed by using a SHIMADZU GC-2014 equipped with an HR-SS-10 (0.25φ×25 m) (SHINWA CHEMICAL INDUSTRIES, LTD.).

### 4. Measurement of Biomass (Dry Weight of Cells)

On each day, 10 mL of culture solution cultured with phosphorus-deficient medium was collected, transferred to a 50-mL tube, and centrifuged at 4670 G and 25° C. for 10 minutes. After the centrifugation, the supernatant was removed with care not to remove any cell. The residual precipitate was suspended by adding H₂O, and the suspension was transferred to a 1.5-mL tube the weight of which had been measured. Centrifugation was performed at 7000 G and 25° C. for 10 minutes. The supernatant was removed with care not to remove any cell, and the 1.5-mL tube was placed in a high-temperature dryer, and dried at 105° C. for 5 hours with the cap removed. The 1.5-mL tube was taken out of the high-temperature dryer, and the biomass was weighed with an electronic balance.

### 5. Method for Creating Lipase-Disrupted Strain

For gene disruption of the class 3 lipase genes No3LIP14 and No3LIP7, a genome editing tool called transcriptional activator-like effector (TALE) nucleases (TALENs) was used. A TALEN is an artificial nuclease obtained by fusing a TALE domain, which is a DNA-binding protein derived from a plant pathogen that enables free design of a target, and a FokI nuclease domain derived from a marine bacterium. The TALE domain has 16 to 18 TALE repeats each consisting of 34 amino acids, and one TALE repeat identifies one nucleotide of DNA. A pair of two TALENs, L-TALEN and R-TALEN, is capable of introducing one DNA double-strand break. In the present experiment, a Platinum TALEN (PtTALEN) system, produced by modifying the amino acids in the fourth and 32nd TALE repeats to give further enhanced activity, was used. TALEN target sequences for the genes were searched for by using a web tool called TAL Effector Nucleotide Targeter 2.0 (https://tale-nt.cac.cornell.edu). Two PtTALEN target sequences were designed for NoLIP14, and PtTALEN pairs corresponding to the target sequences were constructed by means of the Golden gate method. The cleavage activities of the PtTALENs were examined with cultured cells, and LIP14_A (L-TALEN: TCAGTCTGCGGCATGCCC spacer: TTGTGTCGGGCGCGC L-R-TALEN: CAGCCGCCGTGGCTGCGA), from which higher activity was detected, was used to construct an all-in-one PtTALEN vector for expression in Nannochloropsis, the vector was introduced into Nannochloropsis by electroporation, genomic DNA was extracted from the resulting colony, and parts around the target sequence were PCR-amplified to examine mutations in the target sites by direct sequencing. As a result, two No3LIP14 frameshift mutants (6B-6 strain (13 nucleotides deleted), 6B-10 strain (17 nucleotides deleted)) were obtained. For No3LIP7, two PtTALENs were first constructed and checked for activity in cultured cells, and after that mutation of Nannochloropsis was attempted, but no mutation introduced was detectable. Then, two additional PtTALEN target sequences were designed, giving an all-in-one PtTALEN vector targeting LIP7_D (L-TALEN: TTCCAGCAGCAGCCAGTA spacer: TCATCGTCGCTCACC R-TALEN: ACCAAGCCCGCAGCACCA), and two frameshift mutants (3-4-1 strain (4 nucleotides deleted), 3-12-1 strain (2 nucleotides inserted) were obtained therewith.

The sequences used for mutation are set forth in SEQ ID NOs: 27 to 32 in the sequence listing.

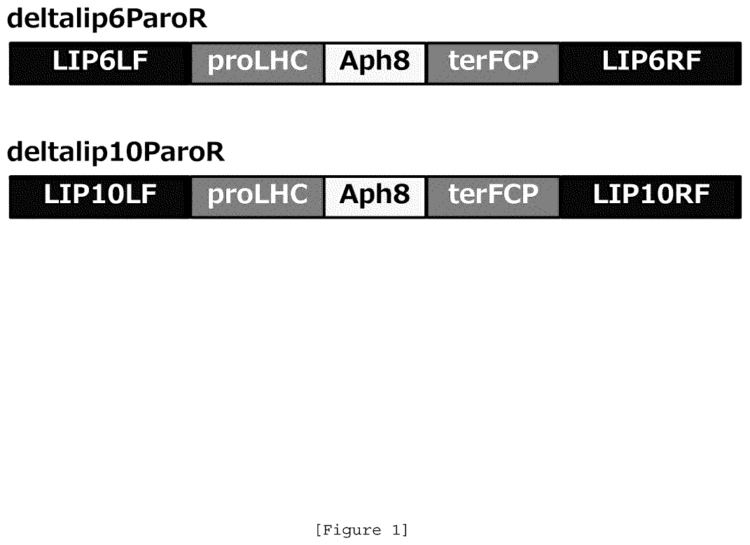

Gene disruption of the class 3 lipase genes No3LIP6 and No3LIP10 were performed by homologous recombination. With the genome of N.2145 as a template, the promoter region of LHC, proLHC, the terminator region of FCP, terFCP, the upstream region of No3LIP6, LIP6LF, the downstream region of No3LIP6, LIP6RF, the upstream region of No3LIP10, LIP10LF, and the downstream region of No3LIP10, LIP10RF, were amplified by PCR. For selection of transgenic strains, a paromomycin resistance gene (Aph8 derived from Streptomyces rimosus) was connected to be sandwiched between proLHC and terFCP; thus proLHCAph8terFCP was formed. proLHCAph8terFCP was connected to be sandwiched between LIP6LF and LIP6RF to produce a construct for No3LIP6 gene disruption, deltalip6ParoR, and connected to be sandwiched between LIP10LF and LIP10RF to produce a construct for No3LIP10 gene disruption, deltalip10ParoR (FIG. 1).

Used for the PCR was a solution obtained by suspending 10 μL of 5×PrimeSTAR GXL Buffer, 4 μL of dNTP Mixture (2.5 mM each), 1 μL of 10 μM primerF, 1 μL of 10 μM primerR, 1 μL of genome solution, 1 μL of PrimeSTAR GXL DNA Polymerase, and 32 μL of sterilized ion-exchanged water.

The PCR was performed in the following reaction cycles.
Step 1: 94° C. for 2 min
Step 2: 30 cycles of 98° C. for 10 sec, 60° C. for 15 sec, and 68° C. for 1 min/kb
   The primers used were as follows.

| | |
|---|---|
| LIP6LF_F | CTGGTAGATGGGCAGGTGTGAG |
| LIP6LF_R | AAGTTAACACAACGAAGCGCCG |
| LIP6RF_F | GCCTGACTTGCCCCAATCCTAC |
| LIP6RF_R | GGAACAGGAGCTTCATATTC |
| LIP10LF_F | GGTGAGATAATGGGGCAAATGC |
| LIP10LF_R | GGTCTTGGCGCCTCCGTTTGCG |
| LIP10RF_F | TGGAGCCCGGACGTTTAGAGAC |
| LIP10RF_R | CACCTCTTCATCTCAGGTTGACC |
| proLHC_F | GGTGGAGTGAGATAGCAGGAGCAT |
| proLHC_R | GCTTGGGAAAGAAGGAGGGAGTTG |
| terFCP_F | GCCGCAGCCTCTTGGGTGAAGTGT |
| terFCP_R | AATACAACCGAAAAGAATAAGGAG |

The sequences used for mutation are set forth in SEQ ID NOs: 33 to 44 in the sequence listing.

### 6. Method for Producing Construct for DGAT Overexpression

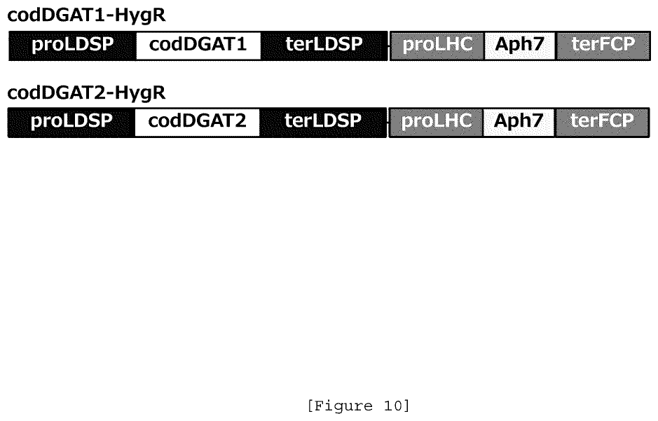

With the genome of N.2145 as a template, the promoter region of LDSP, proLDSP, the terminator region of LDSP, terLDSP, the promoter region of LHC, proLHC, and the terminator region of FCP, terFCP, were amplified by PCR. The codDGAT1 or codDGAT2 gene was connected to be sandwiched between proLDSP and terLDSP; thus, proLDSPcodDGAT1terLDSP or proLDSPcodDGAT2terLDSP was formed. For selection of transgenic strains, a hygromycin resistance gene (Aph7 derived from Streptomyces hygroscopicus) was connected to be sandwiched between proLHC and terFCP; thus, proLHCAph7terFCP was formed. proLDSPcodDGAT1terLDSP or proLDSPcodDGAT2terLDSP was connected to the upstream region of proLHCAph7terFCP to form a construct for TAG production system enhancement, codDGAT1HygR or codDGAT2HygR (FIG. 10).

Used for the PCR was a solution obtained by suspending 10 μL of 5×PrimeSTAR GXL Buffer, 4 μL of dNTP Mixture (2.5 mM each), 1 μL of 10 μM primerF, 1 μL of 10 μM primerR, 1 μL of genome solution, 1 μL of PrimeSTAR GXL DNA Polymerase, and 32 μL of sterilized ion-exchanged water.

The PCR was performed in the following reaction cycles.
Step 1: 94° C. for 2 min
Step 2: 30 cycles of 98° C. for 10 sec, 60° C. for 15 sec, and 68° C. for 1 min/kb
   The primers used were as follows.

| | |
|---|---|
| proLDSP_F | GTCTCTAAGATGGAGTGGATGGAG |
| proLDSP_R | TGTTGATGCGGGCTGAGATTGGTG |
| terLDSP_F | GAAAGATCCAAGAGAGACGAGTAG |
| terLDSP_R | TAAGCTCACCGGCTTTTCTTACAC |
| proLHC_F | GGTGGAGTGAGATAGCAGGAGCAT |
| proLHC_R | GCTTGGGAAAGAAGGAGGGAGTTG |
| terFCP_F | GCCGCAGCCTCTTGGGTGAAGTGT |
| terFCP_R | AATACAACCGAAAAGAATAAGGAG |

The sequences of the primers used for PCR are set forth in SEQ ID NOs: 45 to 52 in the sequence listing.

### 7. Method for Gene Transfer by Electroporation Technique

Gene transfer into Nannochloropsis was performed by an electroporation technique. For the electroporation, an ELEPO21 (Nepa Gene Co., Ltd.) was used.

Nannochloropsis was subjected to gyratory culture with F2N liquid medium at 30 μmol photons/m²/sec, 23° C., and 120 min⁻¹ for 7 to 10 days, successively seeded in 400 mL of F2N liquid medium to a cell concentration of 2 to 3×10⁶ cells/mL, and further cultured. The number of cells cultured for 2 to 4 days in the logarithmic growth phase was counted to confirm to be around 1×10⁷ cells/mL. The culture solution was centrifuged at 4° C. and 5500×g for 10 minutes, and the cells were recovered by precipitation. To the precipitate, ice-cooled 375 mM sorbitol was added, and centrifugation was again performed at 4° C. and 5500×g for 10 minutes to wash the cells as desalting treatment. Cell washing with sorbitol solution was performed four times, and the cells were then suspended in sorbitol solution to adjust to 1×10¹⁰ cells/mL. To give a total volume of 70 μL, 20 μL of the cell suspension solution, 1 μL of DNA to be introduced (1 to 0.1 μg/μL), and 49 μL of 375 mM sorbitol were added and mixed, and the resultant was transferred to a cuvette of 0.1 mm width. The cuvette was left to stand for 10 minutes, and then ice-cooled for 5 minutes. Moisture on the ice-cooled cuvette was wiped off, bubbles in the cuvette were eliminated, the cuvette was then set in a chamber, and an Ω button was pressed to measure the resistance value. For the electroporation, the following conditions were set.

Poring pulse: voltage of 2000 V, pulse width of 5 ms, pulse interval of 50 ms, 1 cycle, polarity of +Transfer pulse: voltage of 150 V, pulse width of 50 ms, pulse interval of 50 ms, 5 cycles, polarity of +/−

Immediately after the electroporation, the cell solution was taken out of the cuvette with a syringe, transferred to a 15-mL Corning tube containing 5 mL of F2N medium, and suspended. The lid of the Falcon tube was loosened and fixed with surgical tape, the side face was doubly wrapped around with a paper towel to make weak-light conditions (approximately 5 μmol photons/m²/sec), and recovery culture was performed with shaking for 48 hours. Top Agar was autoclaved and allowed to cool down to about 60° C., a 5-mL portion thereof was taken and transferred to a 50-mL Corning tube and cooled, and then added to the culture solution and mixed together, and immediately after that the whole volume was spread on an F2N plate with addition of antibiotics for selection to solidify the agar. The Top Agar used was F2N liquid medium containing 0.4% agar. When the agar had solidified, static culture was performed at 20 μmol photons/m²/sec and 23° C. for 3 to 6 weeks for selection against drugs. Colonies that had appeared on the drug-containing plates were each transferred onto a new plate with a sterilized toothpick, and used as transformed strains.

### Experimental Results

### 1. Comparison of Growth and TAG for No3LIP7 Gene Disruption

Nannochloropsis is known to accumulate carbon sources as TAG in intracellular lipid droplets even under normal culture. It is known that the amount of TAG accumulated further increases under phosphorus-deficient conditions. In view of this, 3-4-1 strain and 3-12-1 strain, which are No3LIP7 gene-disrupted strains, were compared with the wild strain on growth and amounts of TAG accumulated under normal culture conditions and those under phosphorus-deficient conditions. For culture, F2N medium was used as a medium for normal culture, and F2N medium removed of Na₂HPO₄ was used as phosphorus-deficient medium. Culture solution was subjected to gyratory culture with an Erlenmeyer flask, and the cell density at the initiation of culture was set to 3×10⁶ cells/mL.

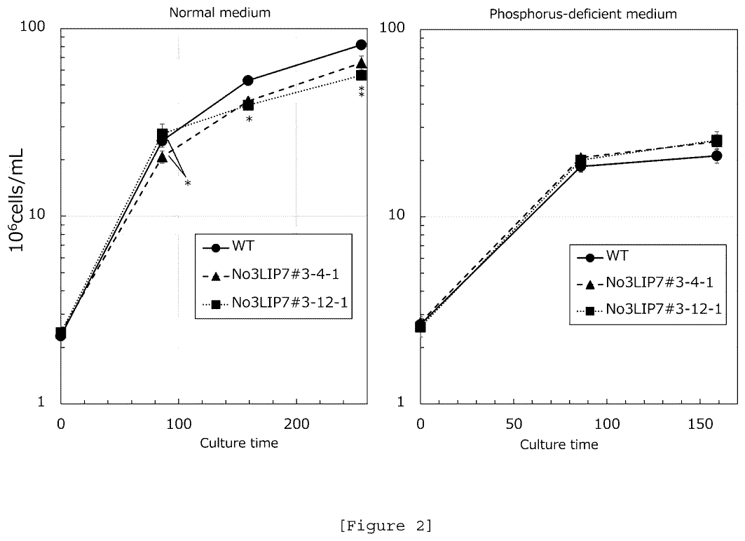

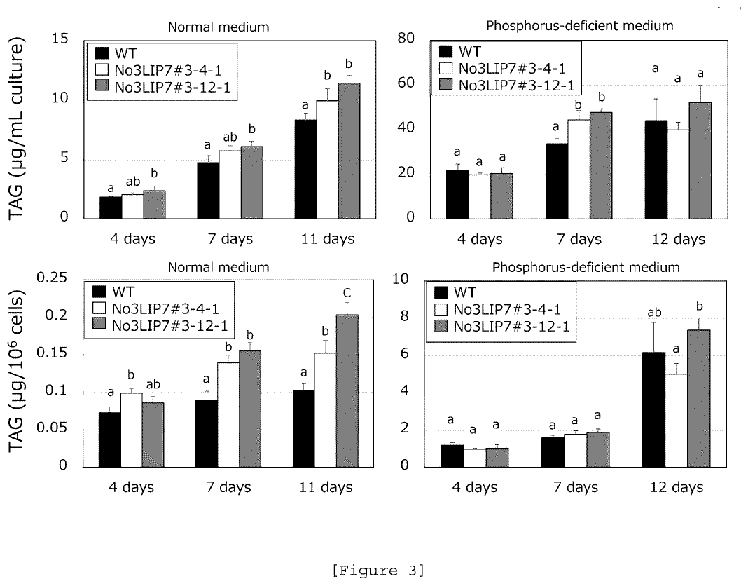

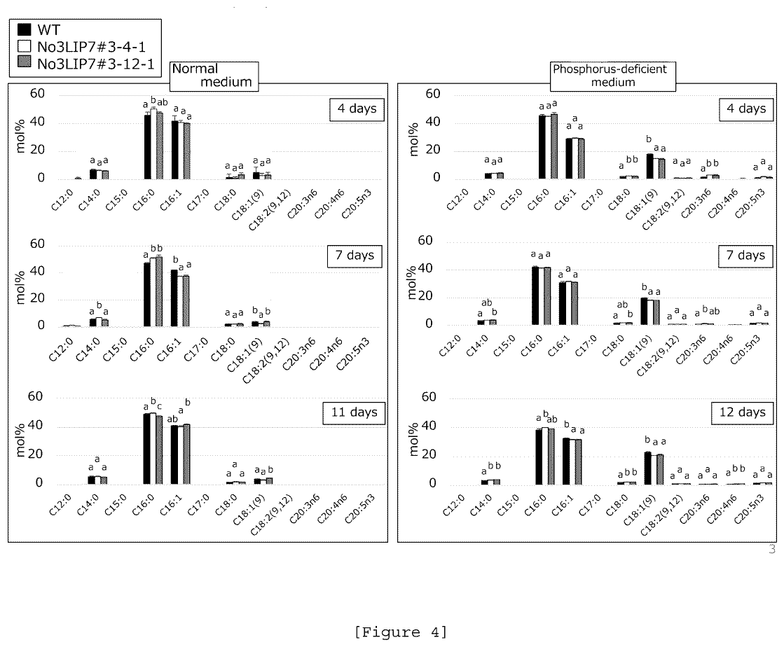

As shown in the left of FIG. 2, the growth of the gene-disrupted strains with normal medium was slightly impaired compared with the wild strain, but, as shown in the right of FIG. 2, no significant difference was found for the growth with phosphorus-deficient medium. As shown in the left of FIG. 3, it was discovered that the amounts of TAG accumulated per culture solution in the gene-disrupted strains with normal medium were larger than those in the wild strain even in the late stage of culture, on day 11. Through comparison of the amounts of TAG accumulated per cell, it was also discovered that those in the gene-disrupted strains were larger than those in the wild strain even in the late stage of culture. In the case of phosphorus-deficient medium, which causes increase in the amounts of TAG accumulated, it was discovered that, as shown in the right of FIG. 3, the amounts of TAG accumulated per culture solution in the gene-disrupted strains with normal medium were larger than those in the wild strain on day 7 of culture, but no significant increase from the wild strain was found in the amounts of TAG accumulated per cell in the gene-disrupted strains. Because the amounts of unsaturated fatty acids in TAG affect the storage life of TAG and the quality of hydrogenated fuels, the fatty acid compositions in TAG in the gene-disrupted strains were compared with those in the wild strain. In the case of normal medium, it was discovered that, as shown in the left of FIG. 4, the rate of C16:1 was lower and the rate of C16:0 was higher than those in the wild strain on day 7 of culture. In the case of phosphorus-deficient medium, as shown in the right of FIG. 4, it was discovered that the rate of C18:1 was lower than those in the wild strain on day 4, 7, and 12 of culture.

### 2. Comparison of Growth and TAG for No3LIP14 Gene Disruption

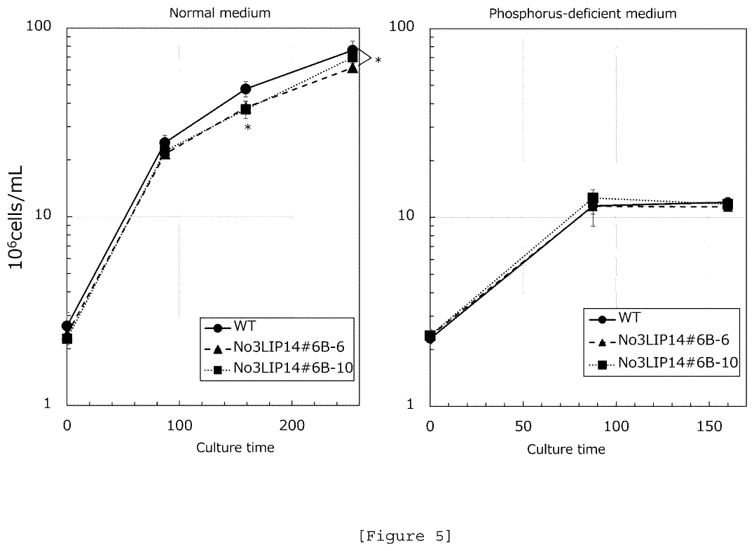

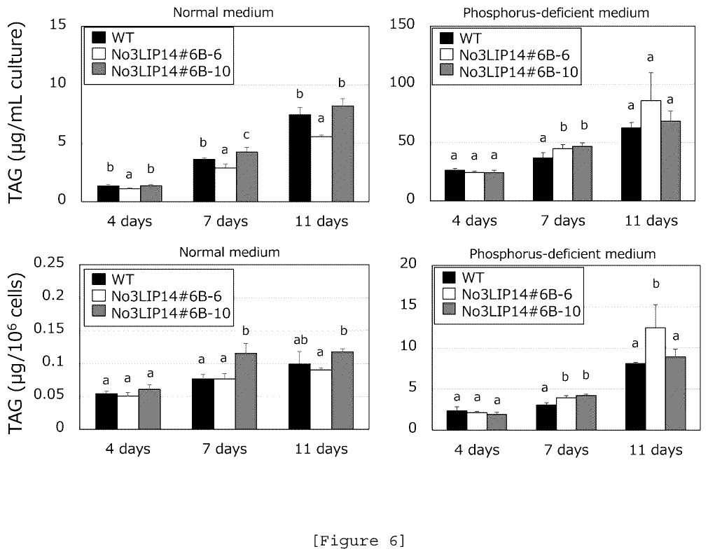

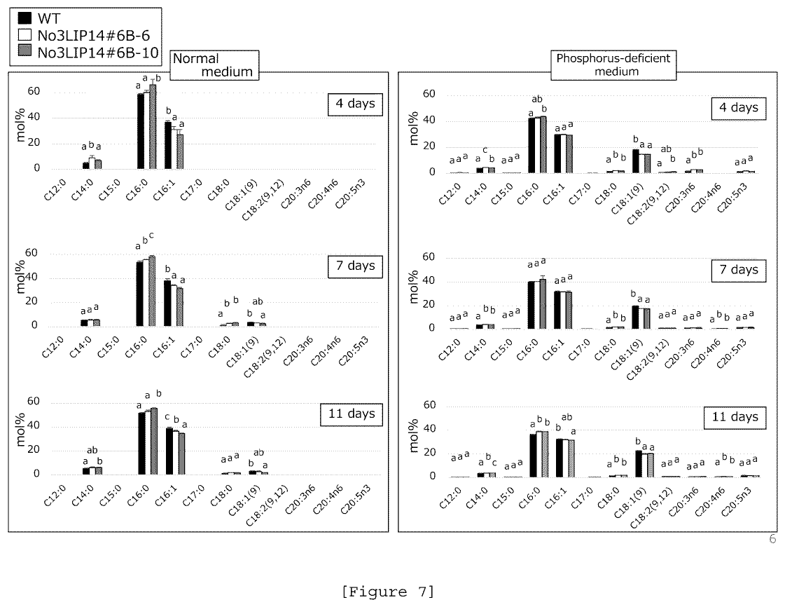

As with the case of the No3LIP7 gene-disrupted strains, No3LIP14 gene-disruptive 6B-6 strain and 6B-10 strain were compared with the wild strain on the growth and amounts of TAG accumulated under normal culture conditions and those under phosphorus-deficient conditions. As shown in the left of FIG. 5, the growth of the gene-disrupted strains with normal medium was impaired compared with the wild strain in the late stage of culture, but, as shown in the right of FIG. 5, no significant difference was found for the growth with phosphorus-deficient medium. As shown in the left of FIG. 6, it was discovered that the amounts of TAG accumulated per culture solution in the gene-disrupted strains with normal medium were larger than those in the wild strain on day 4 and 7 of culture, and the amounts of TAG accumulated per cell in the gene-disrupted strains were larger than those in the wild type in the late stage of culture, on day 7 and 11. In the case of phosphorus-deficient medium, which causes increase in the amounts of TAG accumulated, it was discovered that, as shown in the right of FIG. 6, the amounts of TAG accumulated per culture solution in the gene-disrupted strains were larger than those in the wild strain on day 7, and the amounts of TAG accumulated per cell in the gene-disrupted strains were larger than those in the wild type in the late stage of culture, on day 7 and 11. Through comparison of the fatty acid compositions in TAG in the gene-disrupted strains with those in the wild strain, it was discovered that, as shown in the left of FIG. 7, the rate of C16:1 was lower and the rate of C16:0 was larger in the gene-disrupted strains than those in the wild type in the case of normal medium. In the case of phosphorus-deficient medium, it was discovered that, as shown in the right of FIG. 7, the rates of C18:0 and C18:1 were lower and the rate of C16:0 was higher in the gene-disrupted strains than those in the wild strain.

### Comparison of Growth and TAG for No3LIP6 Gene Disruption

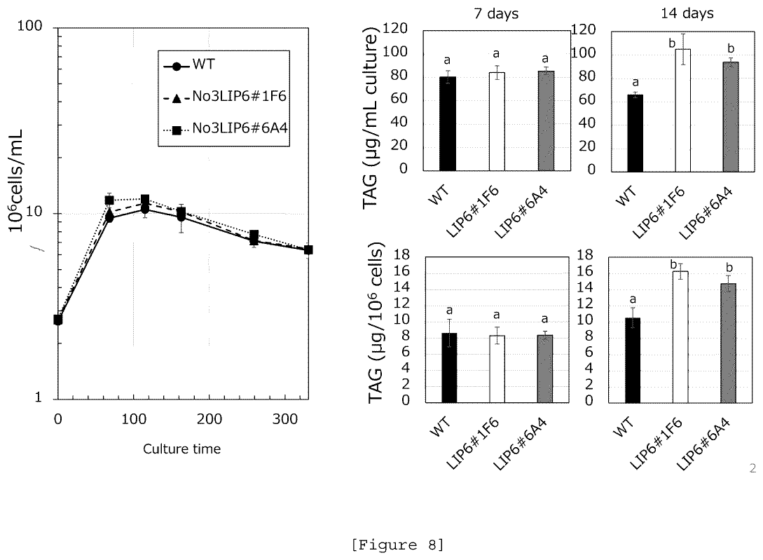

As with the case of the No3LIP7 gene-disrupted strains and No3LIP14 gene-disrupted strains, No3LIP6 gene-disruptive 1F6 strain and 6A4 strain were compared with the wild strain on the growth and amounts of TAG accumulated under phosphorus-deficient conditions. As shown in the left of FIG. 8, no significant difference was found in the growth with phosphorus-deficient medium. As shown in the upper right of FIG. 8, it was discovered that the amounts of TAG accumulated per culture solution in the gene-disrupted strains with phosphorus-deficient medium were larger than those in the wild strain in the late stage of culture, on day 14 of culture, and, as shown in the lower right of FIG. 8, the amounts of TAG accumulated per cell in the gene-disrupted strains were larger than those in the wild strain on day 14.

### Comparison of Growth and TAG for No3LIP10 Gene Disruption

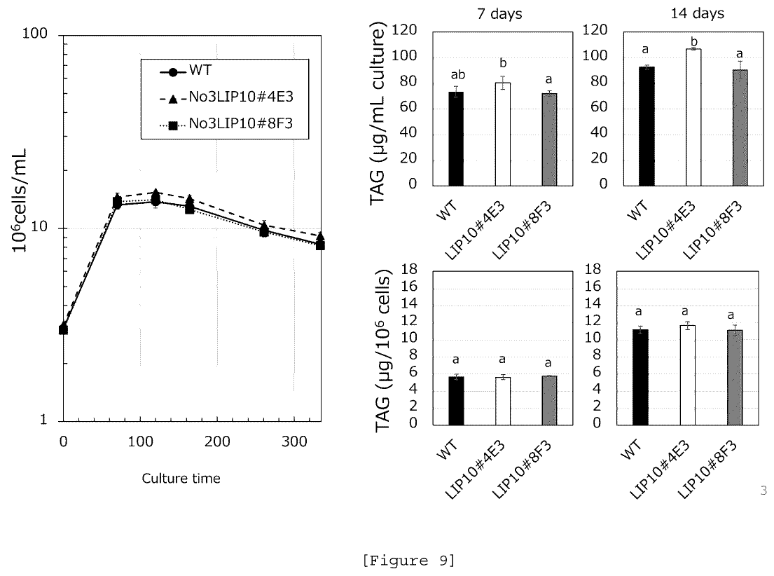

As with the case of the No3LIP7 gene-disrupted strains and No3LIP14 gene-disrupted strain, No3LIP10 gene-disruptive 4E3 strain and 8F3 strain were compared with the wild strain on the growth and amounts of TAG accumulated under phosphorus-deficient conditions. As shown in the left of FIG. 9, no significant difference was found in the growth with phosphorus-deficient medium. As shown in the upper right of FIG. 9, it was discovered that the amounts of TAG accumulated per culture solution in gene-disruptive 4E3 strain with phosphorus-deficient medium were higher than those in the wild strain in the late stage of culture, on day 14 of culture. As shown in the lower right of FIG. 9, no significant difference was found between the amounts of TAG accumulated per cell in the gene-disrupted strains and those in the wild strain.

3. Production of Strains with Enhanced DGAT Expression for Further Enhanced TAG Accumulation

The present inventors have succeeded in causing increase in the amounts of TAG accumulated by disrupting the No3LIP7 gene, No3LIP14 gene, No3LIP6 gene, and No3LIP10 gene. The No3LIP14 gene-disrupted strain and No3LIP6 gene-disrupted strain exhibited more significant increase in TAG accumulation in the late stage of phosphorus-deficient culture than the No3LIP7 gene-disrupted strain and No3LIP10 gene-disrupted strain. In contrast to the No3LIP6 gene-disrupted strain, No3LIP7 gene-disrupted strain, and No3LIP10 gene-disrupted strain, the No3LIP14 gene-disrupted strain exhibited a reduced rate of C16:1 and an enhanced rate of C16:0 in TAG on any day of culture under normal culture, and exhibited reduced rates of C18:0 and C18:1 under phosphorus-deficient culture; these results suggest reduction in the rate of unsaturated fatty acids in TAG, and this is expected to lead to the prolongation of the storage life of TAG and the quality improvement of hydrogenated fuels. Accordingly, in order to achieve further enhanced TAG accumulation, gene transfer for enhanced TAG synthesis was conducted with use of No3LIP14 #6B-6, which exhibited remarkable increase in TAG accumulation in the late stage of phosphorus-deficient culture, as a parent strain. Diacylglycerol acyltransferase (DGAT) is an enzyme that catalyzes the final step of TAG biosynthesis. DGAT is an enzyme prevalent among animals and plants, and two DAGT enzymes, DGAT1 and DGAT2, have been reported. The present inventors have previously found the promoter pSQD2a, which induces high expression under phosphorus-deficient conditions, and developed a method of promoting TAG accumulation in the algal bodies of Chlamydomonas under phosphorus-deficient conditions by linking pSQD2a to the DGTT4 gene, a DGAT2 gene of Chlamydomonas, to transfect Chlamydomonas (Japanese Patent Laid-Open No. 2014-68638). In addition, the present inventors have developed a method of promoting TAG accumulation in the algal bodies of Nannochloropsis by introducing a product obtained by linking pSQD2a to the DGTT4 gene into Nannochloropsis (International Publication No. WO 2015/137449). In the present study, focusing on DGAT1 and DGAT2 of fish feeding on algae as genes to introduce, the present inventors have obtained two genes, codDGAT1 and codDGAT2, by artificial synthesis. With the genome of N.2145 as a template, the promoter region of LDSP, proLDSP, the terminator region of LDSP, terLDSP, the promoter region of LHC, proLHC, and the terminator region of FCP, terFCP, were amplified by PCR. The codDGAT1 or codDGAT2 gene was connected to be sandwiched between proLDSP and terLDSP; thus, proLDSPcodDGAT1terLDSP or proLDSPcodDGAT2terLDSP was formed. For selection of transgenic strains, a hygromycin resistance gene (Aph7 derived from Streptomyces hygroscopicus) was connected to be sandwiched between proLHC and terFCP; thus, proLHCAph7terFCP was formed. A construct for TAG production system enhancement, codDGAT1HygR or codDGAT2HygR, was formed by connecting proLDSPcodDGAT1terLDSP or proLDSPcodDGAT2terLDSP to the upstream region of proLHCAph7terFCP (FIG. 10). No3LIP14 #6B-6 was transfected with the constructs in FIG. 10 by the electroporation technique, and 88 stains were obtained for each through selection by hygromycin. For codDGAT1/LIP14 having codDGAT1HygR introduced thereinto, genomes were recovered from 22 strains of the 88 strains, and PCR confirmed 16 strains to be transfected. The strains confirmed to be transfected were subjected to static culture in a small scale with 2 mL of phosphorus-deficient medium, and it was discovered that the amounts of TAG accumulated per cell in 9 strains of the 16 transfected strains were larger than those in the parent strain LIP14TALEN. For codDGAT2/LIP14 having codDGAT2HygR introduced thereinto, genomes were recovered from 26 strains of the 88 strains, and PCR confirmed 20 strains to be transfected. The strains confirmed to be transfected were subjected to static culture in a small scall with 2 mL of phosphorus-deficient medium, and it was discovered that the amounts of TAG accumulated per cell in 16 strains of the 20 transfected strains were larger than those in the parent strain LIP14TALEN. Two strains for each, four strains in total, with particularly large amounts of TAG accumulated and decent growth were selected as strains with enhanced codDGAT expression: codDGAT1/LIP14 #B7-3, codDGAT1/LIP14 #B8-4, codDGAT2/LIP14 #B1-4, and codDGAT2/LIP14 #B5-4.

4. Comparison of Growth and TAG for codDGAT1/LIP14 and codDGAT2/LIP14

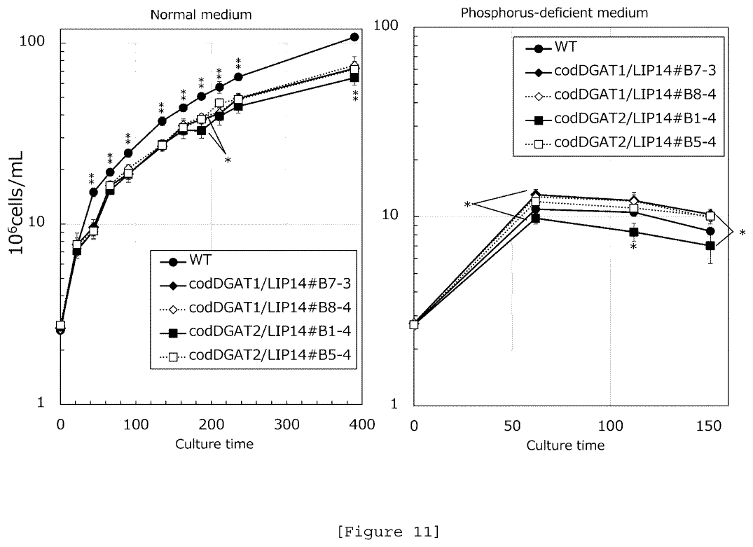

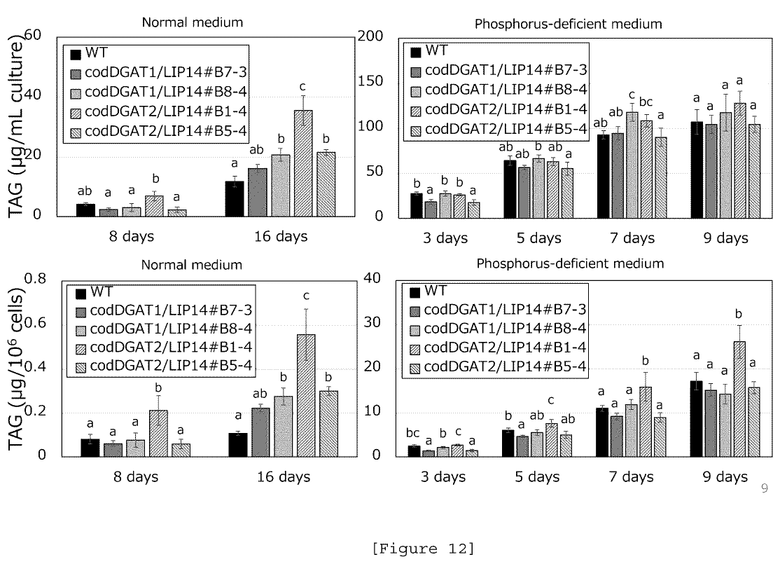

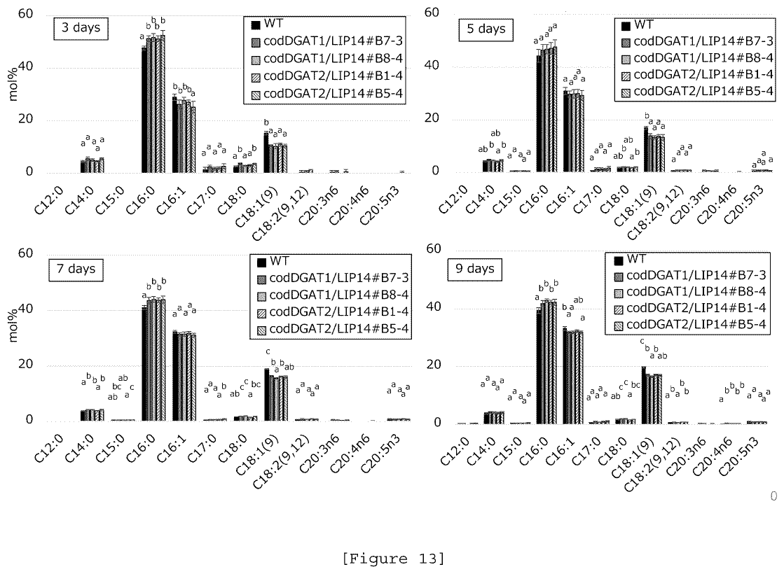

codDGAT1/LIP14 #B7-3, codDGAT1/LIP14 #B8-4, codDGAT2/LIP14 #B1-4, and codDGAT2/LIP14 #B5-4 were compared with the wild strain on the growth and amounts of TAG accumulated under normal culture conditions and those under phosphorus-deficient conditions. In the case of normal medium, as shown in the left of FIG. 11, the strains having codDGAT1 introduced therein and strains having codDGAT2 introduced therein exhibited impaired growth compared with the wild strain; in the case of phosphorus-deficient medium, as shown in the right of FIG. 11, codDGAT2/No3LIP14TALEN #B1-4 exhibited impaired growth than the other strains. As shown in the left of FIG. 12, it was discovered that, in the case of normal medium, the amounts of TAG accumulated per culture solution in the strains having codDGAT introduced therein were higher in the later stage of culture, and the amounts of TAG accumulated in codDGAT2/No3LIP14TALEN #B1-4 reached three times those in the wild strain. Comparison of the amounts of TAG accumulated per cell also discovered that the amounts of TAG accumulated in the strains having codDGAT introduced therein were higher in the later stage of culture, and the amounts of TAG accumulated in codDGAT2/No3LIP14TALEN #B1-4 reached more than five times those in the wild strain. It was discovered that, in the case of phosphorus-deficient medium, which tends to cause TAG accumulation, the amounts of TAG accumulated per culture solution in codDGAT1/No3LIP14TALEN #B7-3 and codDGAT2/No3LIP14TALEN #B1-4 were larger than those in the wild strain on day 7, and the amounts of TAG accumulated per cell in codDGAT2/No3LIP14TALEN #B1-4 were larger than those in the wild strain after day 5. The fatty acid compositions in TAG in the strains having codDGAT introduced therein with phosphorus-deficient medium, which causes increase in the amounts of TAG accumulated, were compared with those in the wild strain, and, as shown in FIG. 13, discovered to have lower rates of C18:1 and higher rates of C16:0 than those in the wild strain through the whole period from day 3 to day 9.

### 5. Comparison of Growth and TAG for High-Density Aerobic Culture System

For putting practical use, amounts of biomass and amounts of TAG accumulated under intense-light conditions and high-density conditions are also important to prepare for outdoor culture. In view of this, the present inventors made comparison on biomass and TAG accumulation with phosphorus-deficient medium under high-density aerobic culture conditions. In high-density aerobic culture, HD medium was used as a medium for normal solution culture. Aeration was performed with 2% CO₂ at 450 mL/min, and the cell density at the initiation of culture under phosphorus deficiency was set to 2×10⁸ cells/mL. Because of difference from the previous culture conditions, culture was performed while the light intensity and temperature were finely controlled as shown in FIG. 14.

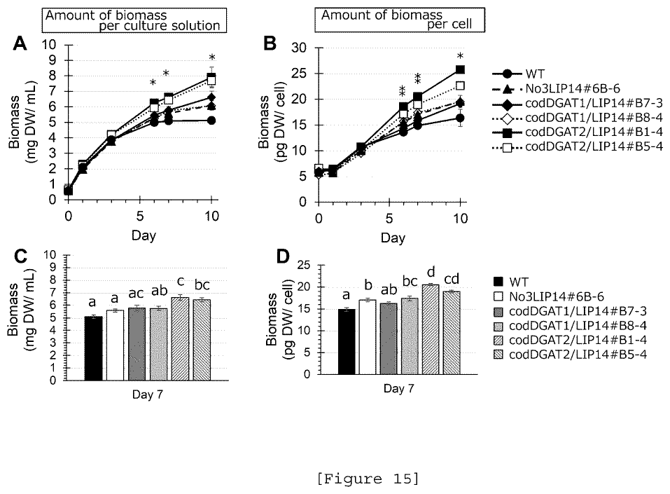

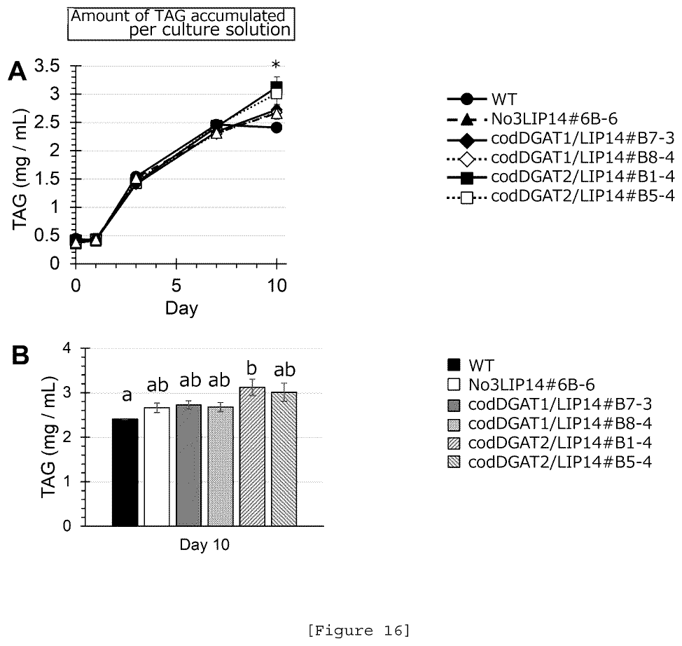

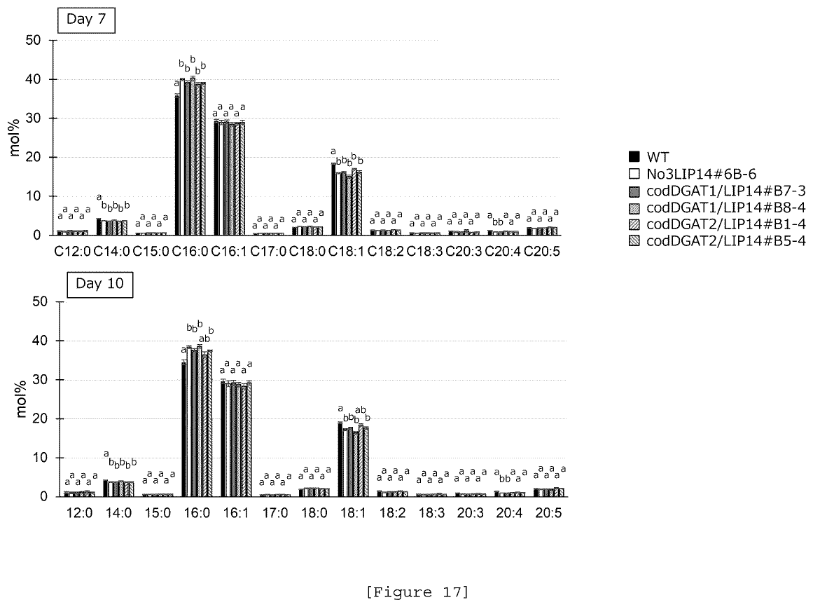

As shown in FIG. 15, the amounts of biomass of the strains having codDGAT2 introduced therein per culture solution and per cell were significantly larger than those of the wild strain and the parent strain (No3LIP14 #6B-6) on day 6, 7, and 10 of culture under phosphorus-deficient conditions. The amounts of biomass of codDGAT1/LIP14 #B8-4 strain per cell were larger than those of the wild strain on day 6 and 7 of culture under phosphorus-deficient conditions. As shown in FIGS. 16A and 16B, the amounts of TAG accumulated in the codDGAT2/LIP14 #B1-4 strain per culture solution were larger than those in the wild strain on day 10 of culture under phosphorus-deficient conditions. From these results, it was discovered that, even under high-density aerobic culture conditions, the combination of LIP14 gene disruption and codDGAT1 or codDGAT2 gene expression results in improvement in the amounts of biomass and amounts of TAG accumulated. The fatty acid compositions in TAG in the strains having codDGAT introduced therein were compared with those in the wild strain, and discovered, as shown in FIG. 17, to have lower C18:1 rates and higher C16:0 rates in the late stage of culture, on day 7 and 10, even under high-density aerobic culture conditions.

### 6. Summary

The present inventors have succeeded in achieving increase in the amounts of TAG accumulated in the late stage of culture under both normal culture and phosphorus-deficient culture conditions by disrupting the newly discovered TAG degradation genes: No3LIP7, No3LIP14, No3LIP6, and No3LIP10. In addition, the present inventors have succeeded in achieving further increase in the amounts of TAG accumulated by introducing the TAG synthesis gene codDGAT1 or codDGAT2 into the TAG degradation gene-disrupted strain No3LIP14 #6B-6. In particular, codDGAT2/No3LIP14 #B1-4 has succeeded in accumulating TAG in amounts five times or more those in the wild strain in the late stage of normal culture, and 1.5 times those in the wild strain in the late stage of phosphorus-deficient culture. It has been discovered that improvement in the amounts of biomass and amounts of TAG accumulated is achieved even under high-density aerobic culture conditions, which are close to those in practical industrial use. It has been also discovered that, under phosphorus-deficient conditions, the strains obtained by introducing codDGAT into No3LIP14 #6B-6 produces TAG having 1%- to 3%-lower rates of C16:1, 3%- to 5%-lower rates of C18:1, and 3%- to 5%-higher rates of C16:0 than the wild strain produces. It has been discovered that, even under high-density aerobic culture conditions, 3%- to 5%-reduced rates of C18:1 and 3%- to 5%-enhanced rates of C16:0 are produced. These results indicate reduction in the rate of unsaturated fatty acids contained in TAG, and this is expected to lead to the prolongation of the storage life of TAG and the quality improvement of hydrogenated fuels. Furthermore, C16, which is more suitable for diesel oil materials than C18, is produced more, and this is expected to lead to increase in the proportion of diesel oils produced from TAG. The present invention, combinations of disruption of novel lipase genes and high DGAT gene expression, is a very useful technique for achieving production cost reduction in production of biofuels and useful lipid production with algae, which have larger biomass than plants, in particular, with Nannochloropsis.

### INDUSTRIAL APPLICABILITY

The present invention is applicable to industrial fields relating to fuels and others.

1. An alga having the following characteristics (1) and (2):
(1) having reduced expression of a class 3 lipase gene; and
(2) having an exogenous diacylglycerol acyltransferase gene introduced or having enhanced expression of an endogenous diacylglycerol acyltransferase gene.

2. The alga according to claim 1, wherein the alga is an alga belonging to the genus Nannochloropsis.

3. The alga according to claim 1, wherein the class 3 lipase gene is a gene encoding the following protein (a), (b), or (c):
(a) a protein consisting of an amino acid sequence set forth in SEQ ID NO: 2, 4, 6, or 8;
(b) a protein consisting of an amino acid sequence derived by substitution, addition, or deletion of 1 to 50 amino acid residues from an amino acid sequence set forth in SEQ ID NO: 2, 4, 6, or 8 and having lipase activity; and
(c) a protein consisting of an amino acid sequence having a homology of 40% or more with an amino acid sequence set forth in SEQ ID NO: 2, 4, 6, or 8 and having lipase activity.

4. The alga according to claim 1, wherein the exogenous diacylglycerol acyltransferase gene is a gene encoding the following protein (a), (b), or (c):
(a) a protein consisting of an amino acid sequence set forth in SEQ ID NO: 10 or 12;
(b) a protein consisting of an amino acid sequence derived by substitution, addition, or deletion of 1 to 50 amino acid residues from an amino acid sequence set forth in SEQ ID NO: 10 or 12 and having diacylglycerol acyltransferase activity; and
(c) a protein consisting of an amino acid sequence having a homology of 40% or more with an amino acid sequence set forth in SEQ ID NO: 10 or 12 and having diacylglycerol acyltransferase activity.

5. The alga according to claim 1, wherein the endogenous diacylglycerol acyltransferase gene is a gene encoding the following protein (a), (b), or (c):
(a) a protein consisting of an amino acid sequence set forth in SEQ ID NO: 14, 16, 18, 20, 22, or 24;
(b) a protein consisting of an amino acid sequence derived by substitution, addition, or deletion of 1 to 50 amino acid residues from an amino acid sequence set forth in SEQ ID NO: 14, 16, 18, 20, 22, or 24 and having diacylglycerol acyltransferase activity; and
(c) a protein consisting of an amino acid sequence having a homology of 60% or more with an amino acid sequence set forth in SEQ ID NO: 14, 16, 18, 20, 22, or 24 and having diacylglycerol acyltransferase activity.

6. The alga according to claim 1, wherein, when cultured under phosphorus-deficient conditions, the alga produces triacylglycerol having a lower rate of oleic acid and a higher rate of palmitic acid than a wild strain produces.

7. A method of producing triacylglycerol, comprising: culturing the alga according to claim 1 to allow the alga to produce triacylglycerol, and collecting the triacylglycerol produced.

8. The method of producing triacylglycerol according to claim 7, wherein the culturing the alga occurs under phosphorus-deficient conditions.


SEQUENCE LISTING (52 sequences)

SEQ ID NO: 1 - DNA, 2175 bp, Nannochloropsis oceanica
  source 1..2175: mol_type = genomic DNA
  CDS 1..2172
atgggggcttctgtcttacgcagactggtgctgcttttactgccgctgcatggagcaactttcttcctttccagcagcagccagtatcatcgtcgctcaccaccaagcccgcagcaccatcgacactcttatccactgcacgccactaccgacgaaatactcaccgccaccgcccctaagcccacgcctgacctctgcatctttgatgctcccaaggccgttcgcctggccgccttttcctttgctacttacggagatcccactggctcccgatggctgcgtctgcccgacggcactgaccttggcttccaatctgatgacttggtgcgcaacaactttaaaagtttgctgttggttcacatcctgcaagcccgcaacctgccagaagaacggggcgggcttattgagagggctctctcaggctttagctgcgacccctacgtcatcacggccgtcaacaattcgtggggtgcagatatcgccatcactaaggccaagagtgctaattccaatcccgattttttccagcgctttttcttgtacgtggaagatcccaacacaacggatctagagctaactgtcaaggataagaacctgctgaaaggcgatgatgttattgggcactgtctggtgcccttgacgcagctgctcgacgggtcaaaaggagagggcgcacctggtcaagaaggcgtgatggagtgggcggggaccttgcaaatcgaaagcgtgcacaaggattttaaggatgctggcaccattgacgtagagctgcagctggtgcccttgaatggagaggccgtgaacaaggtcgaatacgactccaatgaccaggacgtcgccgagagcgacgccgatggcagtggcagcaagcatggtaaattcagtgcacggggtccgaaaagtgtgcttgaaggcattgattggaatgagctgggcgcccgtgtcggtggagtggccagggggctgggccgctaccagtttgcctgctttctgaccaacgaggagaccgatacgcaggggggaatctggtttgatgccaaagccaaggagatcattgtctccttccgaggcacgcagatggaatggaaggatttgctgactgacatggcgatctatcaagaagggttggatgggcctgaggacccgcggttggtgcacgcgggctttcgacgtgcttttcgttctattcaaggcggggtcctacaggccttgaattttatagcttccaatttggtggaggatggatggtctatagacgtgtgtgggcactccttagggggggcactggcgtgcttgatggcccacgagatcgatcggcgcatgcctgccttggtagaggaagggaggctgaatgttttttcctttggggcgccaagggtgggtaacacggcgttttgtgaggactatgacaagcggcttaaaaaccagagctttcgaatagtgaatgggttggacgttgttgcgcgaatgccacgagggtcgggtgaatcttccctattgatggaatacagtcattgcgggcgcacggtactactgaccgagacagaagacggtcgatttagtgtatgggtggagggcgaaagtgaaggcacttgcccactcaaagaaggagaggcatttacgagcacgacgcctacgagtcaatccatggcggagattatcaagaaggccgcaatagaagcgatggagagagggaagggaggtcttgagtttgaaactccatttagcaaggcaacctttgcgaacggtacagctgcctcagtgcaagaaatgctgcgggccgccggaaactttacaaaccttcaagctatgctctcctctacgtcctccatcgactacttctcctctttcacctccattccttcctcgccttctctggggccatttggacagctgagcttgcaaacggatttgcagcaaatcgccaatgcagtgtttggaagcgcgatgaactcgacagctacgattggcaagagcttggggttgaatgagacgtttgtagatagtgagttgagtttaattcgagggttggtgtacggagatgcattagcccaccatatggagccgctttattatgttgcgcttgagaaagcgtttgggattgcgatggaggaaggggtggatgacaagaaaggggaagagaagggggatggtgcaacgaagaggtacggataa

SEQ ID NO: 2 - protein, 724 aa, Nannochloropsis oceanica
MGASVLRRLVLLLLPLHGATFFLSSSSQYHRRSPPSPQHHRHSYPLHATTDEILTATAPKPTPDLCIFDAPKAVRLAAFSFATYGDPTGSRWLRLPDGTDLGFQSDDLVRNNFKSLLLVHILQARNLPEERGGLIERALSGFSCDPYVITAVNNSWGADIAITKAKSANSNPDFFQRFFLYVEDPNTTDLELTVKDKNLLKGDDVIGHCLVPLTQLLDGSKGEGAPGQEGVMEWAGTLQIESVHKDFKDAGTIDVELQLVPLNGEAVNKVEYDSNDQDVAESDADGSGSKHGKFSARGPKSVLEGIDWNELGARVGGVARGLGRYQFACFLTNEETDTQGGIWFDAKAKEIIVSFRGTQMEWKDLLTDMAIYQEGLDGPEDPRLVHAGFRRAFRSIQGGVLQALNFIASNLVEDGWSIDVCGHSLGGALACLMAHEIDRRMPALVEEGRLNVFSFGAPRVGNTAFCEDYDKRLKNQSFRIVNGLDVVARMPRGSGESSLLMEYSHCGRTVLLTETEDGRFSVWVEGESEGTCPLKEGEAFTSTTPTSQSMAEIIKKAAIEAMERGKGGLEFETPFSKATFANGTAASVQEMLRAAGNFTNLQAMLSSTSSIDYFSSFTSIPSSPSLGPFGQLSLQTDLQQIANAVFGSAMNSTATIGKSLGLNETFVDSELSLIRGLVYGDALAHHMEPLYYVALEKAFGIAMEEGVDDKKGEEKGDGATKRYG

SEQ ID NO: 3 - DNA, 2262 bp, Nannochloropsis oceanica
  source 1..2262: mol_type = genomic DNA
  CDS 1..2259
atgcccttgtgtcgggcgcgccagccgccgtggctgcgacagcgggcaccaattgtggttttgcttgtcctgcttttctgtttgctcaatcccgtcgtctcgtcatcactgcgggaaggacggcagcagcagcagcagcagcagcagtcgcaggaggtggaggagacaagaatacgtgaaaataccaagcaggagccggctgaggccatggtagagatcgatgcactgggccagcagcagcagcagcagcagcagcacgagcgagaggagcagttcacagcacaatcaccacccaagcccctccaacgcgaccgagaaggcgctggcattgcccgcctcctacaaagatggctagacgaagaggatggagcaacgaggcgagatcttcatgataatgaggaggagcataaagacaaggacatagaaaaagaggagaatgatgcgttagccttagctgacctaggcaacagcacaggcaacttacaggtatacaaagcccttcgggaaactgctgcggcccataatattacggcccgcgatttttggactttgcaacgtttgattttaacactgtcttttaatagatatgggaacaagccgaccatcttcccgatgtgtgaaatatataaaatagtggggcctttgcggttgcattgtgtcttcattgtctcgtcgtatcggttttacacggcgcagccgccgtcgttgaggaaggtggcgactgaacgctcgtacagtatttatagcggaggagagcaggaggagaagcagcagcaggagcaggagccaccacacgttttacaagacatcggtgtggtgcctgaattcatcgttgacaaacctttaattgagaaggaacggcctgtctatcgatggtgctggcggcagattgtaaatatgtatggctttacgtatcggtggaatgatgaaggagtagaagcggcgacgatggaacagcagcaacacacccaagagggagaaacaagggacaaagatggtggtgctcctccacctccctgcaatggtaaagagaacagcagcagcagtagcagcactgacagcgctcccctccttcctcccgaacctggctatgggaatcagagcctgctgtgggtggaggggccaatggacattccctttcgacggcccattggtcaagtatttacctacttccctcccccttgcagtagtagtagtggtactggttccagaaataggagtagcaggagtgatggtgaacaaaagtacgaggggagaggaggaggacatgtagagggaggaggtcctgagctggtggttgtgataagagggacattatacagtgaggaatggacggctaattttaggttgcagcgggcagatgaggatgaggtggagaagtatgattttgagggtattatggtaccgaagggtttttttgggttatttactgaagtacttccgcaggtcaatgatgcccttgtcctccacggcaacagccacatcactcggctgacactcaccggccattcactgggcgccagcattgcgcttctcctcgcggcatatttcggtcaaaaaagacccgacttaaaaatatccctcataacatttggtgggcccaacaccgtcgataacatattgtttgaaaactactggaaccatcaaataaatgtgcgccaagtgttttatatcggctcggggctcgtgcctcgggacgagagagacagatggttggagggagagagagaagagagacggcagcagcagcagcagtacgagtatcaggatcgtgaacaaccttctgattcccacttgagctttgggatcggggacatggttgcacagatgcccccagactgtactgctgtatccccctgtcccttgctcctcttccacgccaccaatggtctgacgaccaatgatgaccgcagtacaagtcccagtggcttcgatattgaaaacaatttcttcacgcgtctcaggctggcgcgtccgccggggtcgattgttttttatgcttacgacttaaagggacgtggctctgtgagcacatggttggcgtacgaagactttttgacgcatgtctggacaagattagatcgtggtgtaatttcaagtcatgtttgcgcgtacgcgtgttgggtatcttcgctgggaggtggaaagaatggcgaagaaacgcgatgttttttcccggaggaaggggagggacgaggcttgggggagatctgtgatttacaattgacgcgaggtgagcgtttgggaaaaacgcatctttaa

SEQ ID NO: 4 - protein, 753 aa, Nannochloropsis oceanica
MPLCRARQPPWLRQRAPIVVLLVLLFCLLNPVVSSSLREGRQQQQQQQQSQEVEETRIRENTKQEPAEAMVEIDALGQQQQQQQQHEREEQFTAQSPPKPLQRDREGAGIARLLQRWLDEEDGATRRDLHDNEEEHKDKDIEKEENDALALADLGNSTGNLQVYKALRETAAAHNITARDFWTLQRLILTLSFNRYGNKPTIFPMCEIYKIVGPLRLHCVFIVSSYRFYTAQPPSLRKVATERSYSIYSGGEQEEKQQQEQEPPHVLQDIGVVPEFIVDKPLIEKERPVYRWCWRQIVNMYGFTYRWNDEGVEAATMEQQQHTQEGETRDKDGGAPPPPCNGKENSSSSSSTDSAPLLPPEPGYGNQSLLWVEGPMDIPFRRPIGQVFTYFPPPCSSSSGTGSRNRSSRSDGEQKYEGRGGGHVEGGGPELVVVIRGTLYSEEWTANFRLQRADEDEVEKYDFEGIMVPKGFFGLFTEVLPQVNDALVLHGNSHITRLTLTGHSLGASIALLLAAYFGQKRPDLKISLITFGGPNTVDNILFENYWNHQINVRQVFYIGSGLVPRDERDRWLEGEREERRQQQQQYEYQDREQPSDSHLSFGIGDMVAQMPPDCTAVSPCPLLLFHATNGLTTNDDRSTSPSGFDIENNFFTRLRLARPPGSIVFYAYDLKGRGSVSTWLAYEDFLTHVWTRLDRGVISSHVCAYACWVSSLGGGKNGEETRCFFPEEGEGRGLGEICDLQLTRGERLGKTHL

SEQ ID NO: 5 - DNA, 1461 bp, Nannochloropsis oceanica
  source 1..1461: mol_type = genomic DNA
  CDS 1..1458
atgaccctaaccatcagcagcaggccaggtagagcgtcgcgggcctcgaggagaagacggctctgcttcgcggccacgctcgtgatgagcgccgtcggtggcacagcggccctacaccaaccacaccgcccttttgatgccgaggttacgcccgaagggggaactcgggagctcaaactcgacgaggccctggcgggagagtggggtttccggcttctggagccattcatggtctacaatgaaagtcatgtcacgccccagaatcatgacccatggtttccgggctgccctgtctttgacaacctcaacataaacgcccactttggctgccactttatccgactctgcaaagatgtcttgactgctttttgggtagattttagcaagactgccagtaccacgcgacccctgaacccgaacggtctcctgccgcagatcctcgccgataatcgaccgaatccaccgcgccaagtggaccgaactaccgtctacgccgagcgccccacttactactactgctaccgactgatcttactcatgtactctattgcctttgaatacgagaggagcaacatgcaatatagcctgccaagactcgtagacgactgggatcaggagcgctggtacatggtcaattatctcaaaccgcgaaacatcaaccggattgagcttaagggacgccagtcacctatagcagtttccatgatgaaaggtgaccggatgatgattgtgattcgtggtacgatttggacggaggagtggttcaaggactttcagtacaagtgggcgtatgatccggagaacctgttcccaggcaagacacacgaaggcatgtactctgtattcatcgatttttataagggactggaggaagaagtcgaattaaaaaagcccacacacgtcttggtaacgggccacagcttaggggcaggattggcgaatatgatcgggttgcgattggagaacaacatgcaacacaatatgcgcgtggacgtggtgggatttgggggccccaacactggagacatcgagtttgccgaggtgttccgcaagaccgtcaatagccgacatattattttcctaggtcgcgggaatgagccgcactcgcacatgtacacggcaggcgacattactgcgcaatacacctgcgagccttatcctggatgcgacatcctgtccacgggtccctttgggtatttctaccattacattcgcccgcacaaccaagtccccttctatgccgaagaaatgccgaatccaaagaagtggaacgaagtctccaacattaggcgcatcattccttcgaccaagggggtgattgcttcgcatctatgtagttacatttgctggacttctcaggctgtcggggatttggacagccggtgctattttgcaaatgagccgggccggcctgacttgccccaatcctactattgcgacgtgagtcaggttcccatatag

SEQ ID NO: 6 - protein, 486 aa, Nannochloropsis oceanica
MTLTISSRPGRASRASRRRRLCFAATLVMSAVGGTAALHQPHRPFDAEVTPEGGTRELKLDEALAGEWGFRLLEPFMVYNESHVTPQNHDPWFPGCPVFDNLNINAHFGCHFIRLCKDVLTAFWVDFSKTASTTRPLNPNGLLPQILADNRPNPPRQVDRTTVYAERPTYYYCYRLILLMYSIAFEYERSNMQYSLPRLVDDWDQERWYMVNYLKPRNINRIELKGRQSPIAVSMMKGDRMMIVIRGTIWTEEWFKDFQYKWAYDPENLFPGKTHEGMYSVFIDFYKGLEEEVELKKPTHVLVTGHSLGAGLANMIGLRLENNMQHNMRVDVVGFGGPNTGDIEFAEVFRKTVNSRHIIFLGRGNEPHSHMYTAGDITAQYTCEPYPGCDILSTGPFGYFYHYIRPHNQVPFYAEEMPNPKKWNEVSNIRRIIPSTKGVIASHLCSYICWTSQAVGDLDSRCYFANEPGRPDLPQSYYCDVSQVPI

SEQ ID NO: 7 - DNA, 2094 bp, Nannochloropsis oceanica
  source 1..2094: mol_type = genomic DNA
  CDS 1..2091
atgcggcaacgcaggcggaggctgtcggcggtgtttgcggccggcagcctgacgttgctggtcttgttgagcatgggccaaggagctattgccgacatcgccgccggtgccgggcagactggcagatccgcccacaccacgaaaatagccttaatggaaatgcctgattctgtaggtccgctgaggagcaaggacaagatcatcccctcggtggcattggaggatgtgtggatagagagggagaaagaggaagaggatgaggaagttcatgtcgaaggaaacctaattcccgcgccattaccggcgaaagccacgagcgaaaaaaagttgcgacagctggatagacaagagaagactactacttcatctcctcccaccaaggccgccacagcaaccgtaggagcggcagcccctctagacccagcagtcgttgttcccaccaaggaaacccgtatattaccctgggagacgaaagcatggcaacgagatcttgacgaactcaatgccttgcttgacatggacattgaacctgccgccattaacacagaggcctcatccctcgctgccgagattgggagtgacgggcactttgagactatgggtagtatgggaaatcttatgaacctcctctccccctcaccagaccctgcttggcctttctgctcccctgttggcatcctgtataaaacaggattggtttgcccggtcatgaatattgcggccaagattctgatgaggtctcccccggactttgagaaattgacaacggagcatcattgtcctaaccctactggggagctcccagaacagttggaggagctgagggaaggtggtttgccgatgccgatgattattcccaaggaggtggtggagagggagaagcctacatatgcgtggtatttgacgcatgtttacaatatgtacgcctttgccttctcctacgcggacgacatcctcagcacccacagcaacgcagactaccacacgctccgccccctaaagcccaagggatgggagaaccaaaacgtgtacttcgttaaggaccccgaaggttggaattggcccatggggcagtcactcacgaaggggaatagcatggtggtcttcttctgcggaaccatgttcccgggcggatgggaggcagactttaagacgagctttgcggcgaaatcggagacggagaagacgggtttgccgggccgagtgcatgatggctttttcgacttcgcccagcagtttttcgaggatttcacccgacaggtcgatctttacggtattcaaagcgtgacattcgctggacacagcctgggcggagctatctcacaggtggccgcggccttcatggtcaagcattacagtcacatcaaaattaattgcgtcacattcggatcaccacaggtcggcgatcggaccttcactgatttcttgaacgcccgtgtcaatctccgacgcatggcctacattggatccgggcagcttaatgagccggcagaacgaggccttaattacggcctgggcgacgtgatcactcagatgcctctggcgtgctacaacctctccatgcccttttgctcgcatatgacgtttatcgacaacaaggacgatctctcaagtttccaattcgtcatggcggcgggcaacgttccgatctacgcttccgacctgcgaaatacccaggagtggctgaaaaaggaaaattttctttttggagcccggacgtttagagacatcccagaaggagcaatggcttgtcacatatgctcctatgcgtgctggacctcggccgcaactgacgaccctgacaaccggtgttatttctccgggaagggagggagggagggagggaaggagggagggcaggcggaagggaggtatttgtgcgagattgatttgaccgagagtttgactgctcgttttcccaatttggaagggaatccggttaatagcgacttggtgcgtgaggggtcagtggcagaggggaagggagggggaggaggaggaggaggaggaggaggaggaggaggaggcggaggaggcggcgggttaatggatggaatgttgatgggggggaaaagggggttgaggatcaagtag

SEQ ID NO: 8 - protein, 697 aa, Nannochloropsis oceanica
MRQRRRRLSAVFAAGSLTLLVLLSMGQGAIADIAAGAGQTGRSAHTTKIALMEMPDSVGPLRSKDKIIPSVALEDVWIEREKEEEDEEVHVEGNLIPAPLPAKATSEKKLRQLDRQEKTTTSSPPTKAATATVGAAAPLDPAVVVPTKETRILPWETKAWQRDLDELNALLDMDIEPAAINTEASSLAAEIGSDGHFETMGSMGNLMNLLSPSPDPAWPFCSPVGILYKTGLVCPVMNIAAKILMRSPPDFEKLTTEHHCPNPTGELPEQLEELREGGLPMPMIIPKEVVEREKPTYAWYLTHVYNMYAFAFSYADDILSTHSNADYHTLRPLKPKGWENQNVYFVKDPEGWNWPMGQSLTKGNSMVVFFCGTMFPGGWEADFKTSFAAKSETEKTGLPGRVHDGFFDFAQQFFEDFTRQVDLYGIQSVTFAGHSLGGAISQVAAAFMVKHYSHIKINCVTFGSPQVGDRTFTDFLNARVNLRRMAYIGSGQLNEPAERGLNYGLGDVITQMPLACYNLSMPFCSHMTFIDNKDDLSSFQFVMAAGNVPIYASDLRNTQEWLKKENFLFGARTFRDIPEGAMACHICSYACWTSAATDDPDNRCYFSGKGGREGGKEGGQAEGRYLCEIDLTESLTARFPNLEGNPVNSDLVREGSVAEGKGGGGGGGGGGGGGGGGGGGGLMDGMLMGGKRGLRIK

SEQ ID NO: 9 - DNA, 1515 bp, Gadus morhua
  source 1..1515: mol_type = genomic DNA
  CDS 1..1512
atgggggatagttgcgcgaatgcggccttacggcgccgagcaaccttttccggggttaggtcgctgacggtacaaccagcctctggaggatctgcaactgcccagtttgccctgacagccagcagttctcgtgaacctgggcagccaaacgggctccatacgacccactcaaagtcgaagcacgaaaaggaggagaagcggaaacgcagaaacgatcattcgggccgtttgtcatgccatcaacaccagcaaagtctgttaagctcttcctcaggcttctcgaattatcgaggcatccttaactggtgcgtggtgatgttggtattgtcgaattcgcgattgttccttgagaacctcctcaagtacggaatccttgtcgatcctatccaagtgctgtcgttgttcgttaaggacccctattcatggcccgcagcttgcctcgtcatcgtctccaatgtgttcattttggcggctctgtacacagagaggcagctgtccaaaggttctctctctgaacaggtaggcttactcgtgcattcggtgaacttgtccatcattctgtgctttccagccgttgtggtgttgctcgtgcctagtatctctccagtcggaggagtgtttgctctcgggacctatacgattctcctcttgaagctctacagttacaaggacgtcaataggtggtgcagagagttgagttccatcaaagtgaagacgctgtcccgtagcaactcctgtccttccgctcctcccgtcataggagttgaccagatcgtgtgttatccgggaaacctgacgattagggacctttactacttcgtttttgcgcctacactgtgttatgagctcaactttccccggtccccgaagattcgcatgagctttttgttgcggaggttgttggagatgttgtttctcacccagatgcttgtggcgttgacgcaacagtggatggtgcccatcatccagtcctccatgaaaccccttgaggagatggacttgtcccgtatgatggaacggctcttacggctggcagtacccaatcacttgctttggttgatgtttttctattggtttttccatagcagcctaaacttcaccgctgagctgctgagatttggtgatcggcagttctacaaggattggtggaatgccgaaactgtgacctacttctggcagaattggaacatacctgtgcacaagtggtgtctaaggcacttctattgtcccctactccgcaaagggttcagcaagatggtcagccaaagcgcggtcttctttcttagcgcattcttccacgagtacctgatgagcgtgccgctcaggatgtttcgactgtgggcgtttatgggcatgatggcgcagttaccgttggcatggctcgtaactcgctttcttcgcggcaattacgggaatgccgctgtctggattagcctgataattgggcaaccgattgccgtcctgatgtatgtgcacgactactacgtgctacataacggtcaggagatccattga

SEQ ID NO: 10 - protein, 504 aa, Gadus morhua
MGDSCANAALRRRATFSGVRSLTVQPASGGSATAQFALTASSSREPGQPNGLHTTHSKSKHEKEEKRKRRNDHSGRLSCHQHQQSLLSSSSGFSNYRGILNWCVVMLVLSNSRLFLENLLKYGILVDPIQVLSLFVKDPYSWPAACLVIVSNVFILAALYTERQLSKGSLSEQVGLLVHSVNLSIILCFPAVVVLLVPSISPVGGVFALGTYTILLLKLYSYKDVNRWCRELSSIKVKTLSRSNSCPSAPPVIGVDQIVCYPGNLTIRDLYYFVFAPTLCYELNFPRSPKIRMSFLLRRLLEMLFLTQMLVALTQQWMVPIIQSSMKPLEEMDLSRMMERLLRLAVPNHLLWLMFFYWFFHSSLNFTAELLRFGDRQFYKDWWNAETVTYFWQNWNIPVHKWCLRHFYCPLLRKGFSKMVSQSAVFFLSAFFHEYLMSVPLRMFRLWAFMGMMAQLPLAWLVTRFLRGNYGNAAVWISLIIGQPIAVLMYVHDYYVLHNGQEIH

SEQ ID NO: 11 - DNA, 1086 bp, Gadus morhua
  source 1..1086: mol_type = genomic DNA
  CDS 1..1083
atgaaaaccttgttggcagcctattccggagtcgtcaaaggaacgggttcatccgtgttgtctgtccttcaagatctgccatcccctccttggccttgtcgatcgaagatggagaagcacctacagtgcatttcggtcctccaatgggtgatctccttccttgctctggggttgagcgctactgttctcctcatctacatgttctgtacggattgctggctcatagcggccatgtatactgcgtggttgatcttcgactggaatacacccaaacaaggagggagacgctcgagttgggtacgcaactggacggtctggacctattttcgggactactttcccgtgaggctgatcaagacgcatgaccttctaccgtctcgtaactatatcttcggctatcacccgcatgggatcttttgctttggcgccttttgcaactttggcaccgaagcgacatcgttctccaagaagttccccgggattaagccgagtttggccacattagcgggcaatttccggtttccggtactgcgcgattatctcatgtctggagggatatgccctgtgaacaagaacagcatagactacttgctgacctgcaatggtacgggtaatgccgtggtgattgtggtaggaggagcagcagaatcgcttgagtgtgctcctggaaaaaacagcgttacgttgtcacggaggaaggggtttgtccgtctcgccttagagaaaggctccgatttggtgcccgtgtacagctttggggagaatgacgcataccagcagatcattttcgaggaagggagttggtggaggcttgtccaacgaaagatgcagaagatcgttgggttcgctccttgtctctttcacggctgttccttctttagcgcggagacgtggggtattgtgccctactgtaagcccattaccactgtggttggcgagccaatcaccgtgcccaaagtggaggaaccaacaccggatgtagtggatcagtaccatgccatgtacatccagagcctgcagtgcttgttcgacaagtacaagacccacttcggactcaaggagtctgacgtcctggagattctgtga

SEQ ID NO: 12 - protein, 361 aa, Gadus morhua
MKTLLAAYSGVVKGTGSSVLSVLQDLPSPPWPCRSKMEKHLQCISVLQWVISFLALGLSATVLLIYMFCTDCWLIAAMYTAWLIFDWNTPKQGGRRSSWVRNWTVWTYFRDYFPVRLIKTHDLLPSRNYIFGYHPHGIFCFGAFCNFGTEATSFSKKFPGIKPSLATLAGNFRFPVLRDYLMSGGICPVNKNSIDYLLTCNGTGNAVVIVVGGAAESLECAPGKNSVTLSRRKGFVRLALEKGSDLVPVYSFGENDAYQQIIFEEGSWWRLVQRKMQKIVGFAPCLFHGCSFFSAETWGIVPYCKPITTVVGEPITVPKVEEPTPDVVDQYHAMYIQSLQCLFDKYKTHFGLKESDVLEIL

SEQ ID NO: 13 - DNA, 1314 bp, Nannochloropsis oceanica
  source 1..1314: mol_type = genomic DNA
  CDS 1..1311
atgtctatgcacaaactgactcgaccgagcgtgctgtccattgagtatccctcgcgggattatacgggttatttgaacctggcaatgatcatcctcggtgtccacttttcccacgccgtagtcgactgcgtctcgctggtgtggcggtacggagtgcagctcccgaagcattctctggtggaggtgccctgcctcatgtgcgcgctctccctcaccatcaacatcttcttggcttggttcaccgaatacctcgcctcccgtcgcttcttcccctcctcgatggcggtaggtgtgctgcattccctgaactgcctatggacgctgctctacccctgccacgtggcatggagccggccggacgtgcctctgcacacgttcttgttgctattttggagtgtgatcgccttcttaaagctcgtctcctggtcccacactaattgggatcttcgacatgctttcttctcccggcgcgcccgcaagtctcaggctcacctgccagcagcggcccttcacgaagacggctacaacaatgccaagcccctcgagagcggcgccacgcggtacccgcactcggtctcactaagcaacataagcttcttcttcttctgtcccactctctgctaccagcccgactacccacgcgcgccaacgattcgattgcggacactcgcctcgctcacctttcgtatcatcgtcatgacggcctttgcgggcttcatcatcgaccagcagatccaccccatcatccagaacaccatgagccacgtcgatagcctcgatctcctcaaggccctgggtgagctgctccgccttgccatcccctccaccttcgtctggctcattttcttctacgtctacttccactgcaccctcaatcttttcgcggaattaacgcgtttcggggatcgattgtttttcaaggattggtggaacagcaccagcttctcccgctattggcgaacttggaatcttcccgtccatcagtttgtcgtccgtcatgtgtacttccccttgctacgcgcgggggcgtctaaaatgacggccaacgtcgccgttttcgctgtctcggccttcttccacgagctgctgatctcgataccctgccacgtcgtgcggctgtgggcgtttttggccatgatgggccagatccctctcatctaccttacggaccagctggagaaaaccttgttcaaggaaacgcaggcagggaattacaccttctggcttatcttctgtatcttcggacagccaatggcagtgctcttgtactatgctgacttctctgcccgcactaccagcgagagcgctctttag

SEQ ID NO: 14 - protein, 437 aa, Nannochloropsis oceanica
MSMHKLTRPSVLSIEYPSRDYTGYLNLAMIILGVHFSHAVVDCVSLVWRYGVQLPKHSLVEVPCLMCALSLTINIFLAWFTEYLASRRFFPSSMAVGVLHSLNCLWTLLYPCHVAWSRPDVPLHTFLLLFWSVIAFLKLVSWSHTNWDLRHAFFSRRARKSQAHLPAAALHEDGYNNAKPLESGATRYPHSVSLSNISFFFFCPTLCYQPDYPRAPTIRLRTLASLTFRIIVMTAFAGFIIDQQIHPIIQNTMSHVDSLDLLKALGELLRLAIPSTFVWLIFFYVYFHCTLNLFAELTRFGDRLFFKDWWNSTSFSRYWRTWNLPVHQFVVRHVYFPLLRAGASKMTANVAVFAVSAFFHELLISIPCHVVRLWAFLAMMGQIPLIYLTDQLEKTLFKETQAGNYTFWLIFCIFGQPMAVLLYYADFSARTTSESAL

SEQ ID NO: 15 - DNA, 1092 bp, Nannochloropsis oceanica
  source 1..1092: mol_type = genomic DNA
  CDS 1..1089
atgacgccgcaagccgacatcaccagcaagacgacacccaacctcaagacggctgcgtcatccccctccaagacctcgcccgccccctccgttcaatacaaggcggcgaatggcaaggtgatcacggtggccatggccgagcaagacgacgggaacatgggcattttccgcgagtgttttgcaatggtgacaatgggcataattatgtcgtggtattacatcgtcgtcattctctccctcctctgcttggtggggatctgcatcttccctgcctggcgggcggtagcggccacggtttttgtgcttatgtggagtgcggcgctattgccgcttgactaccagggatgggatgctttctgcaactcctttatcttcaggctgtggcgggactacttccactatgaatacgtcctggaggagatgatcgacccaaacaagcgctacctctttgctgagatgcctcacggtatcttcccctggggagaggtgatttccatttcgatcaccaaacagctttttcccgggagccgcgtaggctccatcggtgcgagtgtcatcttcctccttcccggtctcaggcacttcttcgcttggatcgggtgtcggcccgcgagcccagagaacatcaaaaagatttttgaggatgggcaggactgtgccgtgacggtggggggggtcgccgagatgtttctagtcggaggagacaaggaacgactgtacctgaagaagcacaagggtttcgttcgagaagccatgaagaatggggcggacctggttcctgtcttctgcttcggcaacagcaagctgttcaatgtggtgggggagagcagtcgggtttctatgggcctgatgaagcgcctctcaaggaggattaaggccagcgtcctcatcttttacggccgtctcttcctgcccattccgattcgacacccgctcttgttcgtggtggggaagcccctgccggtcgtgcacaaggcagagccgaccaaggaggagatcgcggcaacgcacgcactcttttgcgagaaggtcgaggagctttactacaaatacaggccggagtgggagacgcgcccgttgtccattgagtaa

SEQ ID NO: 16 - protein, 363 aa, Nannochloropsis oceanica
MTPQADITSKTTPNLKTAASSPSKTSPAPSVQYKAANGKVITVAMAEQDDGNMGIFRECFAMVTMGIIMSWYYIVVILSLLCLVGICIFPAWRAVAATVFVLMWSAALLPLDYQGWDAFCNSFIFRLWRDYFHYEYVLEEMIDPNKRYLFAEMPHGIFPWGEVISISITKQLFPGSRVGSIGASVIFLLPGLRHFFAWIGCRPASPENIKKIFEDGQDCAVTVGGVAEMFLVGGDKERLYLKKHKGFVREAMKNGADLVPVFCFGNSKLFNVVGESSRVSMGLMKRLSRRIKASVLIFYGRLFLPIPIRHPLLFVVGKPLPVVHKAEPTKEEIAATHALFCEKVEELYYKYRPEWETRPLSIE

SEQ ID NO: 17 - DNA, 1083 bp, Nannochloropsis oceanica
  source 1..1083: mol_type = genomic DNA
  CDS 1..1080
atggccaaggccaacttcccaccctcggcacgctatgttaatatgacgcaggtctatgcgacaggtgctcacaatatgccggacgaggaccgtctcaaggtgatgaatggactttccaagcccttgacggaggccaagcccggtgatttggggtttggggatgtggagtccatgacgttctgtgaagagtttgtagcgattatgttcttattgatcattgtcgggagcatgctttggataccgattgcagtgttgggtttcgccctgtatgtccgcagcgcaatggcgtgggtggtgatgctcatcgtgttcttcaccctgagcctgcacccagtcccgcgcatccatgatatggttcattccccattgaatcacttcatattcaagtacttcagtctaaaaatggcgagtgatgcaccactggatagtgctgggcgctatatttttgtcgctccgccacatggcgtgctgcctatggggaatcttatgacggtgcacgcgatgaaggcgtgtggtggattggagttccgtgggctgacgactgatgtcgcgcttaggctgcctttgtttcgacactatttaggcgctattggtactattgccgcgacgaggcacgtggcgaagcagtacctcgacaaaggatggtcaataggtatatcttcgggcggagtcgcggagattttcgaagttaacaataaggatgaggtggtgttgatgaaggagcgaaagggctttgtgaagcttgcccttcgcacggggactccgttggtagcttgttatatttttgggaataccaagctgttgtcggcgtggtatgatgatgggggggtgttggagggcctgtcgcgttatttgaaatgtggtgtattgccactttggggtcgctttggtttgccgcttatgcaccgtcatcctgtattgggcgcgatggcaaagccgatcgtagttcccaaggtggagggagagcccacgcaggagatgattgatgagtaccatagtctcttctgtcagacgctggtcgatctatttgatagatacaagaccttgtatggctggccggacaagaagctgcttattaagtga

SEQ ID NO: 18 - protein, 360 aa, Nannochloropsis oceanica
MAKANFPPSARYVNMTQVYATGAHNMPDEDRLKVMNGLSKPLTEAKPGDLGFGDVESMTFCEEFVAIMFLLIIVGSMLWIPIAVLGFALYVRSAMAWVVMLIVFFTLSLHPVPRIHDMVHSPLNHFIFKYFSLKMASDAPLDSAGRYIFVAPPHGVLPMGNLMTVHAMKACGGLEFRGLTTDVALRLPLFRHYLGAIGTIAATRHVAKQYLDKGWSIGISSGGVAEIFEVNNKDEVVLMKERKGFVKLALRTGTPLVACYIFGNTKLLSAWYDDGGVLEGLSRYLKCGVLPLWGRFGLPLMHRHPVLGAMAKPIVVPKVEGEPTQEMIDEYHSLFCQTLVDLFDRYKTLYGWPDKKLLIK

SEQ ID NO: 19 - DNA, 1314 bp, Nannochloropsis oceanica; identical to SEQ ID NO: 13
  source 1..1314: mol_type = genomic DNA
  CDS 1..1311

SEQ ID NO: 20 - protein, 437 aa, Nannochloropsis oceanica; identical to SEQ ID NO: 14

SEQ ID NO: 21 - DNA, 1092 bp, Nannochloropsis oceanica
  source 1..1092: mol_type = genomic DNA
  CDS 1..1089
atgacgccgcaagccgacatcaccagcaagacgacacccaacctcaagacggctgcgtcatccccctccaagacctcgcccgccccctccgttcaatacaaggcggcgaatggcaaggtgatcacggtggccatggccgagcaagacgacgggaacatgggcattttccgcgagtgttttgcaatggtgacaatgggcataattatgtcgtggtattacatcgtcgtcattctctccctcctctgcttggtggggatctgcatcttccctgcctggcgggcggtagcggccacggtttttatgcttatgtggagtgcggcgctattgccgcttgactaccagggatgggatgctttctgcaactcctttatcttcaggctgtggcgggactacttccactatgaatacgtcctggaggagatgatcgacccaaacaagcgctacctctttgctgagatgcctcacggtatcttcccctggggagaggtgatttccatttcgatcaccaaacagctttttcccgggagccgcgtaggctccatcggtgcgagtgtcatcttcctccttcccggtctcaggcacttcttcgcttggatcgggtgtcggcccgcgagcccagagaacatcaaaaagatttttgaggatgggcaggactgtgccgtgacggtggggggggtcgccgagatgtttctagtcggaggagacaaggaacgactgtacctgaagaagcacaagggtttcgttcgagaagccatgaagaatggggcggacctggttcctgtcttctgcttcggcaacagcaaactgttcaatgtggtgggggagagcagtcgggtttctatgggcctgatgaagcgcctctcaaggaggattaaggccagcgtcctcatctcttacggccgtctcttcctgcccattccgattcgacacccgctcttgttcgtggtggggaagcccctgccggtcgtgcacaaggcagaaccgaccaaggaggagatcgcggcaacgcacgcactcttttgcgagaaggtcgaggagctttactacaaatacaggccggagtgggagacgcgcccgttgtccattgagtaa

SEQ ID NO: 22 - protein, 363 aa, Nannochloropsis oceanica
MTPQADITSKTTPNLKTAASSPSKTSPAPSVQYKAANGKVITVAMAEQDDGNMGIFRECFAMVTMGIIMSWYYIVVILSLLCLVGICIFPAWRAVAATVFMLMWSAALLPLDYQGWDAFCNSFIFRLWRDYFHYEYVLEEMIDPNKRYLFAEMPHGIFPWGEVISISITKQLFPGSRVGSIGASVIFLLPGLRHFFAWIGCRPASPENIKKIFEDGQDCAVTVGGVAEMFLVGGDKERLYLKKHKGFVREAMKNGADLVPVFCFGNSKLFNVVGESSRVSMGLMKRLSRRIKASVLISYGRLFLPIPIRHPLLFVVGKPLPVVHKAEPTKEEIAATHALFCEKVEELYYKYRPEWETRPLSIE

SEQ ID NO: 23 - DNA, 1041 bp, Nannochloropsis oceanica
  source 1..1041: mol_type = genomic DNA
  CDS 1..1038
atgacgcaggtctatgcgacaggtgctcacaatatgccggacgaggaccgtctcaaggtgatgaatggactttccaagcccttgacggaggccaagcccggtgatttggggtttggggatgtggagtccatgacgttctgtgaagagtttgtagcgattatgttcttattgatcattgtcgggagcatgctttggataccgattgcagtgttgggtttcgccctgtatgtccgcagcgcaatggcgtgggtggtgatgctcatcgtgttcttcaccctgagcctgcacccagtcccgcgcatccatgatatggttcattccccattgaatcacttcatattcaagtacttcagtctaaaaatggcgagtgatgcaccactggatagtgctgggcgctatatttttgtcgctccgccacatggcgtgctgcctatggggaatcttatgacggtgcacgcgatgaaggcgtgtggtggattggagttccgtgggctgacgactgatgtcgcgcttaggctgcctttgtttcgacactatttaggcgctattggtactattgccgcgacgaggcacgtggcgaagcagtacctcgacaaaggatggtcaataggtatatcttcgggcggagtcgcggagattttcgaagttaacaataaggatgaggtggtgttgatgaaggagcgaaagggctttgtgaagcttgcccttcgcacggggactccgttggtagcttgttatatttttgggaataccaagctgttgtcggcgtggtatgatgatgggggggtgttggagggcctgtcgcgttatttgaaatgtggtgtattgccactttggggtcgctttggtttgccgcttatgcaccgtcatcctgtattgggcgcgatggcaaagccgatcgtagttcccaaggtggagggagagcccacgcaggagatgattgatgagtaccatagtctcttctgtcagacgctggtcgatctatttgatagatacaagaccttgtatggctggccggacaagaagctgcttattaagtga

SEQ ID NO: 24 - protein, 346 aa, Nannochloropsis oceanica
MTQVYATGAHNMPDEDRLKVMNGLSKPLTEAKPGDLGFGDVESMTFCEEFVAIMFLLIIVGSMLWIPIAVLGFALYVRSAMAWVVMLIVFFTLSLHPVPRIHDMVHSPLNHFIFKYFSLKMASDAPLDSAGRYIFVAPPHGVLPMGNLMTVHAMKACGGLEFRGLTTDVALRLPLFRHYLGAIGTIAATRHVAKQYLDKGWSIGISSGGVAEIFEVNNKDEVVLMKERKGFVKLALRTGTPLVACYIFGNTKLLSAWYDDGGVLEGLSRYLKCGVLPLWGRFGLPLMHRHPVLGAMAKPIVVPKVEGEPTQEMIDEYHSLFCQTLVDLFDRYKTLYGWPDKKLLIK

SEQ ID NO: 25 - DNA, 767 bp, Nannochloropsis sp.
  source 1..767: mol_type = genomic DNA
gtctctaagatggagtggatggaggaggaggcgagcgtagcagcaagcgtgagttatacagccaggcacatgtcgcaatccttcggtctcgggcttaaaatccacgcactaatcacgctgggccatgcaaagagcaatgccgaggcccaccacacaaaacgctgtgtcgcgcgttgcggcctgaagcttcatacttcttagtcgccgccaaaagggctcgagagacgagacccgttggcatgaccgatgttgttcgacgcggtttgcttcgtcacagtcgacgtgattcaggaatctggagcctgcagatcatttttttcagcctgatatcgttcttttccactgagaaccatcagaccaccttttcttccattgtgtgaaggagtaggagttgccgtgctgctttgtgggagacatctgcgatggtgaccagcctcccgtcgtctggtcgacgtgacgagcctcttcactgttcttcgacggagagacgcaagcgagacggctctagaccttttggacacgcattctgtgtgtgaactagtggacagtgataccacgtctgaaagctcaccactgcccatggtgcagctacttgtcacaaagttttgactccgtcggtatcaccattcgcgctcgtgtgcctggttgttccgccacgccggcctgccccggggcggggcaatattctaaaatctcacgcaaaacaccgcacttacccctcacacatattcgtgatagaccaccaccaatctcagcccgcatcaaca

SEQ ID NO: 26 - DNA, 489 bp, Nannochloropsis sp.
  source 1..489: mol_type = genomic DNA
gaaagatccaagagagacgagtagagattttttttttgggattgatgtttgtcgttctttgagttgtcgtcgagttacgccttttgtaagaatgttccgcaggagaggaggaggatgggcatgagtgagggtgagagggcttgcccgcttttttttttaaaaacgctgaagacgtggttgtcaaacaaaccccccatagaaacgattttgttacggtgcggtccagacgtcacttgaatggctccgcggaaaggccagggagggaaggggggagggaggaaacatgaaacatgttgaacggctcaacagggtttgggggacaagagaggtagcgccctgatggactgctccctcccctcctttccctcaatgtctcattcatccatgcttcccccttctctctctcccctccgttccatcccccgcgggcgtggtagtggcgtgatgggatccactaaaatgtacgtgtaagaaaagccggtgagctta

SEQ ID NO: 27 - DNA, 18 bp, synthetic construct
  misc_feature 1..18: note = Description of Artificial Sequence: TALEN target sequence
  source 1..18: mol_type = other DNA
tcagtctgcggcatgccc

SEQ ID NO: 28 - DNA, 15 bp, synthetic construct
  misc_feature 1..15: note = Description of Artificial Sequence: spacer
  source 1..15: mol_type = other DNA
ttgtgtcgggcgcgc

SEQ ID NO: 29 - DNA, 18 bp, synthetic construct
  misc_feature 1..18: note = Description of Artificial Sequence: TALEN target sequence
  source 1..18: mol_type = other DNA
cagccgccgtggctgcga

SEQ ID NO: 30 - DNA, 18 bp, synthetic construct
  misc_feature 1..18: note = Description of Artificial Sequence: TALEN target sequence
  source 1..18: mol_type = other DNA
ttccagcagcagccagta

SEQ ID NO: 31 - DNA, 15 bp, synthetic construct
  misc_feature 1..15: note = Description of Artificial Sequence: spacer
  source 1..15: mol_type = other DNA
tcatcgtcgctcacc

SEQ ID NO: 32 - DNA, 18 bp, synthetic construct
  misc_feature 1..18: note = Description of Artificial Sequence: TALEN target sequence
  source 1..18: mol_type = other DNA
accaagcccgcagcacca

SEQ ID NO: 33 - DNA, 22 bp, synthetic construct
  misc_feature 1..22: note = Description of Artificial Sequence: primer
  source 1..22: mol_type = other DNA
ctggtagatgggcaggtgtgag

SEQ ID NO: 34 - DNA, 22 bp, synthetic construct
  misc_feature 1..22: note = Description of Artificial Sequence: primer
  source 1..22: mol_type = other DNA
aagttaacacaacgaagcgccg

SEQ ID NO: 35 - DNA, 22 bp, synthetic construct
  misc_feature 1..22: note = Description of Artificial Sequence: primer
  source 1..22: mol_type = other DNA
gcctgacttgccccaatcctac

SEQ ID NO: 36 - DNA, 20 bp, synthetic construct
  misc_feature 1..20: note = Description of Artificial Sequence: primer
  source 1..20: mol_type = other DNA
ggaacaggagcttcatattc

SEQ ID NO: 37 - DNA, 22 bp, synthetic construct
  misc_feature 1..22: note = Description of Artificial Sequence: primer
  source 1..22: mol_type = other DNA
ggtgagataatggggcaaatgc

SEQ ID NO: 38 - DNA, 22 bp, synthetic construct
  misc_feature 1..22: note = Description of Artificial Sequence: primer
  source 1..22: mol_type = other DNA
ggtcttggcgcctccgtttgcg

SEQ ID NO: 39 - DNA, 22 bp, synthetic construct
  misc_feature 1..22: note = Description of Artificial Sequence: primer
  source 1..22: mol_type = other DNA
tggagcccggacgtttagagac

SEQ ID NO: 40 - DNA, 23 bp, synthetic construct
  misc_feature 1..23: note = Description of Artificial Sequence: primer
  source 1..23: mol_type = other DNA
cacctcttcatctcaggttgacc

SEQ ID NO: 41 - DNA, 24 bp, synthetic construct
  misc_feature 1..24: note = Description of Artificial Sequence: primer
  source 1..24: mol_type = other DNA
ggtggagtgagatagcaggagcat

SEQ ID NO: 42 - DNA, 24 bp, synthetic construct
  misc_feature 1..24: note = Description of Artificial Sequence: primer
  source 1..24: mol_type = other DNA
gcttgggaaagaaggagggagttg

SEQ ID NO: 43 - DNA, 24 bp, synthetic construct
  misc_feature 1..24: note = Description of Artificial Sequence: primer
  source 1..24: mol_type = other DNA
gccgcagcctcttgggtgaagtgt

SEQ ID NO: 44 - DNA, 24 bp, synthetic construct
  misc_feature 1..24: note = Description of Artificial Sequence: primer
  source 1..24: mol_type = other DNA
aatacaaccgaaaagaataaggag

SEQ ID NO: 45 - DNA, 24 bp, synthetic construct
  misc_feature 1..24: note = Description of Artificial Sequence: primer
  source 1..24: mol_type = other DNA
gtctctaagatggagtggatggag

SEQ ID NO: 46 - DNA, 24 bp, synthetic construct
  misc_feature 1..24: note = Description of Artificial Sequence: primer
  source 1..24: mol_type = other DNA
tgttgatgcgggctgagattggtg

SEQ ID NO: 47 - DNA, 24 bp, synthetic construct
  misc_feature 1..24: note = Description of Artificial Sequence: primer
  source 1..24: mol_type = other DNA
gaaagatccaagagagacgagtag

SEQ ID NO: 48 - DNA, 24 bp, synthetic construct
  misc_feature 1..24: note = Description of Artificial Sequence: primer
  source 1..24: mol_type = other DNA
taagctcaccggcttttcttacac

SEQ ID NO: 49 - DNA, 24 bp, synthetic construct
  misc_feature 1..24: note = Description of Artificial Sequence: primer
  source 1..24: mol_type = other DNA
ggtggagtgagatagcaggagcat

SEQ ID NO: 50 - DNA, 24 bp, synthetic construct
  misc_feature 1..24: note = Description of Artificial Sequence: primer
  source 1..24: mol_type = other DNA
gcttgggaaagaaggagggagttg

SEQ ID NO: 51 - DNA, 24 bp, synthetic construct
  misc_feature 1..24: note = Description of Artificial Sequence: primer
  source 1..24: mol_type = other DNA
gccgcagcctcttgggtgaagtgt

SEQ ID NO: 52 - DNA, 24 bp, synthetic construct
  misc_feature 1..24: note = Description of Artificial Sequence: primer
  source 1..24: mol_type = other DNA
aatacaaccgaaaagaataaggag